(19) United States
(12) Patent Application Publication
(10) Pub. No.: US 2021/0332113 A1
(43) Pub. Date: Oct. 28, 2021

(54) METHODS OF SCREENING ANTIBODIES FOR TREATING AND/OR PREVENTING NECROTIZING ENTEROCOLITIS (NEC)

(71) Applicant: UNIVERSITY OF PITTSBURGH - OF THE COMMONWEALTH SYSTEM OF HIGHER EDUCATION, Pittsburgh, PA (US)

(72) Inventors: Timothy Wesley Hand, Pittsburgh, PA (US); Michael Jason Morowitz, Pittsburgh, PA (US); Kathyayini Parlakoti Gopalakrishna, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 17/341,272

(22) Filed: Jun. 7, 2021

Related U.S. Application Data
(63) Continuation of application No. PCT/US2019/062725, filed on Nov. 22, 2019.
(60) Provisional application No. 62/778,114, filed on Dec. 11, 2018.

(51) Int. Cl. C07K 16/12; G01N 33/569; A61P 31/04; A61K 9/00; A23L 33/00; A23L 33/10; A23C 9/20; A23C 9/152

(52) U.S. Cl. CPC C07K 16/1228; G01N 33/56911; A61P 31/04; A61K 9/0095; A61K 2039/505; A23L 33/10; A23C 9/206; A23C 9/152; A23L 33/40

(57) ABSTRACT

Provided are antibodies that bind to bacteria associated with necrotizing enterocolitis (NEC), methods of detecting the same, and methods of using the same for treating and/or preventing NEC. The antibodies that bind to bacteria associated with NEC are detected by, e.g., detecting binding of the antibody to a bacterium in a bacterial array that includes the bacteria associated with NEC.

### CROSS-REFERENCE TO RELATED APPLICATION

This application is a continuation of International Patent Application No. PCT/US2019/062725, filed on Nov. 22, 2019, which claims priority to U.S. Provisional Application No. 62/778,114, filed on Dec. 11, 2018, each of which is incorporated herein by reference in its entirety.

### SEQUENCE LISTINGS

The specification further incorporates by reference the Sequence Listing submitted herewith via EFS on Jun. 7, 2021. Pursuant to 37 C.F.R. § 1.52(e)(5), the Sequence Listing text file, identified as 0723960854SL.txt, is 1,173 bytes and was created on Jun. 7, 2021. The Sequence Listing, electronically filed herewith, does not extend beyond the scope of the specification and thus does not contain new matter.

### FIELD

The presently disclosed subject matter relates to antibodies that bind to bacteria associated with necrotizing enterocolitis (NEC), methods of detecting the same, and methods of using the same for treating and/or preventing NEC.

### BACKGROUND OF THE INVENTION

Neonates are particularly susceptible to infection by colonizing micro-organisms, and mammals protect their offspring via antibodies, in particular, immunoglobulin A (IgA), secreted into the breast milk. Necrotizing enterocolitis (NEC) is a disease of newborn premature infants characterized by tissue damage and immunopathology, presumably related to bacterial colonization of the immature intestine. The impact of NEC is immense. Children diagnosed with the disease still have a mortality rate of about 25%. Treatment of the disease often requires multiple surgeries and the cost of treating a single patient can be millions of dollars. Additionally, “successfully” treated patients have often had significant portions of their small intestine removed and often developed long-term consequences (such as failure to thrive) due to absorption issues. Studies have shown that the incidence of NEC is significantly reduced in infants fed with human milk, though the mechanisms underlying this protective benefit are not clear. Therefore, novel and improved methods of treating and/or preventing to this disease are urgently needed.

### SUMMARY OF THE INVENTION

The presently disclosed subject matter provides methods of detecting an antibody and methods for treating or preventing necrotizing enterocolitis (NEC).

In one aspect, the present disclosure features a method of detecting an antibody in a sample including the antibody, the method including: (a) providing a bacterial array including bacteria that are associated with NEC; (b) contacting the bacterial array with the sample including the antibody; and (c) detecting the antibody by detecting binding of the antibody to a bacterium of the bacterial array.

In another aspect, the present disclosure features a method of detecting an antibody in a sample including the antibody, the method including: (a) providing a bacterial array including bacteria that are associated with necrotizing enterocolitis (NEC); (b) isolating an antibody repertoire including the antibody from the sample; (c) contacting the bacterial array with the antibody repertoire; and (d) detecting the antibody by detecting binding of the antibody to a bacterium of the bacterial array.

In certain embodiments, following detecting of the antibody in the sample, the sample is selected to be administered to an infant.

In another aspect, the present disclosure features a method of treating or preventing NEC in an infant in need thereof, the method including: (i) identifying a sample including an antibody that binds to a bacterium associated with NEC, wherein the sample is identified by: (a) providing a bacterial array including bacteria that are associated with NEC; (b) contacting the bacterial array with the sample including the antibody; and (c) detecting the antibody by detecting binding of the antibody to a bacterium of the bacterial array; and (ii) administering the sample including the antibody that binds to the bacterium associated with NEC to the infant in an effective amount to treat or prevent NEC in the infant.

In another aspect, the present disclosure features a method of treating or preventing NEC in an infant in need thereof, the method including: (i) identifying a sample including an antibody that binds to a bacterium associated with NEC, wherein the sample is identified by: (a) providing a bacterial array including bacteria that are associated with NEC; (b) isolating an antibody repertoire including the antibody from the sample; (c) contacting the bacterial array with the antibody repertoire; and (d) detecting the antibody by detecting binding of the antibody to a bacterium of the bacterial array; and (ii) administering the sample including the antibody that binds to the bacterium associated with NEC to the infant in an effective amount to treat or prevent NEC in the infant.

In certain embodiments of any one of the methods disclosed herein, the method further comprises washing the bacterial array before detecting the binding of an antibody to the bacterial array. In some embodiments, the sample includes an antibody library. In further embodiments, the sample is a breast milk. In certain embodiments, the breast milk is human breast milk. In certain embodiments, the antibody is an immunoglobulin, e.g., IgA, IgD, IgE, IgG and IgM. In certain embodiments, the immunoglobulin is an IgA class antibody. In some embodiments, the IgA class antibody is an IgA1 or IgA2 antibody. In further embodiments, the IgA class antibody is a secretory IgA (sIgA) antibody.

In certain embodiments, the immunoglobulin is detected by an anti-immunoglobulin antibody. In certain embodiments, the anti-immunoglobulin antibody comprises a detectable label, e.g., a radioisotope or a fluorescent label. In certain embodiments, the binding of the antibody to the bacterial array is detected by flow cytometry.

In certain embodiments of any one of the methods disclosed herein, the bacterial array comprises bacteria of one or more family selected from the group consisting of Enterobacteriaceae, Pasteurellaceae, Pseudomonadaceae, Tissierellaceae, Veillonellaceae, Peptostreptococcaceae, Lachnospiraceae, Clostridiaceae, Enterococcaceae, Staphylococcaceae, Streptococcoceae and Bifidobacteriaceae.

In certain embodiments of any one of the methods disclosed herein, the bacterial array comprises bacteria of one or more genus selected from the group consisting of Enterobacter, Escherichia, Citrobacter, Salmonella, Klebsiella, Corynebacterium, Lactobacillus, Proteus, Haemophilus, Staphylococcus, Pseudomonas, Clostridium, Bifidobacterium, Enterococcus, Streptococcus and Veillonella.

In certain embodiments of any one of the methods disclosed herein, the bacterial array comprises one or more bacterium selected from the group consisting of E. coli NIHMB, E. coli AIEC 2A, E. coli CUMT8, Enterobacter, Enterococcus, E. coli NIHT5, Citrobacter, Klebsiella, Pseudomonas, Streptococcus, Yersinis, S. aureus (ptnA), Salmonella, S. aureus, Enterobacter (NECteria monoculture), Escherichia (ECORL), Escherichia (ECMB), Escherichia (ECT5), Escherichia (MT8), Escherichia coli 909, Escherichia coli 910, Escherichia coli 4185, Citrobacter rodentium 51459, Salmonella typhimurium 3261, Klebsiella pneumoniae, Enterobacter cloacae, Klebsiella oxytoca, Klebsiella aerogenes, Corynebacterium spp., Lactobacillus casei, Lactobacillus paracasei, Proteus mirabilis, Streptococcus agalactiae, Streptococcus salivarius, Streptococcus pneumoniae 19F, Veillonella parvula, Veillonella atypica, Enterococcus faecalis 19433, Enterococcus faecalis 286, Enterococcus faecium, Bifidobacterium breve, Bifidobacterium bifidum, Clostridium butyricum, Clostridium sporogenes, Clostridium perfringens, Pseudomonas aerginosa 01, Staphylococcus aureus, Staph aureus Protein A-, MSSA NARSA227, MSSA NARSA235, Staphylococcus capitis, Staph epidermidis B1468, Staph epi. NARSA101, Staph epi. NIHLM087, Staph saprophyticus, Haemophilus parainfluenza, and S. epidermidis. In certain embodiments, the bacterial array includes Escherichia coli NIHMB, E. coli AIEC 2A, E. coli CUMT8, Enterobacter, Enterococcus, E. coli NIHT 5, Citrobacter, Klebsiella, Pseudomonas, Streptococcus, Yersinis, Staphylococcus aureus (ptnA), Salmonella, S. aureus, Enterobacter (NECteria monoculture), Escherichia (ECORL), Escherichia (ECMB), Escherichia (ECTS), Escherichia (MT8), E. coli 909, E. coli 910, E. coli 4185, Citrobacter rodentium 51459, Salmonella typhimurium 3261, Klebsiella pneumoniae, Enterobacter cloacae, K. oxytoca, K aerogenes, Corynebacterium spp., Lactobacillus casei, Lactobacillus paracasei, Proteus mirabilis, Streptococcus agalactiae, Streptococcus salivarius, Streptococcus pneumoniae 19F, Veillonella parvula, Veillonella atypica, Enterococcus faecalis 19433, Enterococcus faecalis 286, Enterococcus faecium, Bifidobacterium breve, Bifidobacterium bifidum, Clostridium butyricum, Clostridium sporogenes, Clostridium perfringens, Pseudomonas aerginosa 01, Staphylococcus aureus, Staph aureus Protein A-, MSSA NARSA227, MSSA NARSA235, Staphylococcus capitis, Staph epidermidis B1468, Staph epi. NARSA101, Staph epi. NIHLM087, Staph saprophyticus, Haemophilus parainfluenza, and S. epidermidis

In further embodiments of any one of the methods disclosed herein, the bacterial array includes one or more bacterium selected from the group consisting of C. rodentium, E. aerogenes, E. cloacae, E. coli 587, E. coli 596, E. coli 605, E. coli 909, E. coli 910, E. coli 4185, E. coli ECO2A, E. coli ECMB, E. coli ECTS, E. coli MTB, S. typhimurium SL3261, Enterobacter NECMONO, K. aerogenes 13048, K. oxytoca 43165, K. oxytoca K405, K. pneumoniae, S. marcescens 855, S. marcescens 853, P. mirabilis, P. vulgaris, P. aeruginosa, M. nonliquefaciens, L. casei, S. agalactiae, S. aureus RS, S. aureus TWH, S. capitis, S. epidermidis LM087, S. epidermidis LM088, S. epidermidis RS, S. saprophyticus, E. faecalis 2649, E. faecalis 19433, and E. faecium. In certain embodiments, the bacterial array includes C. rodentium, E. aerogenes, E. cloacae, E. coli 587, E. coli 596, E. coli 605, E. coli 909, E. coli 910, E. coli 4185, E. coli ECO2A, E. coli ECMB, E. coli ECTS, E. coli MTB, S. typhimurium SL3261, Enterobacter NECMONO, K. aerogenes 13048, K. oxytoca 43165, K. oxytoca K405, K. pneumoniae, S. marcescens 855, S. marcescens 853, P. mirabilis, P. vulgaris, P. aeruginosa, M. nonliquefaciens, L. casei, S. agalactiae, S. aureus RS, S. aureus TWH, S. capitis, S. epidermidis LM087, S. epidermidis LM088, S. epidermidis RS, S. saprophyticus, E. faecalis 2649, E. faecalis 19433, and E. faecium.

In certain embodiments of any one of the methods disclosed herein, the infant is a premature infant.

In another aspect, the present disclosure features a method for treating or preventing NEC in an infant in need thereof comprising: administering to the infant an effective amount of milk or an infant formula including an effective amount of an antibody that binds to one or more bacterium from the family selected from the group consisting of Enterobacteriaceae, Streptococcoceae, Veillonellaceae, Enterococcaceae, Bifidobacteriaceae, Clostridiaceae, Pseudomonadaceae, Staphylococcaceae, Pasteurellaceae and any combination thereof. In some embodiments, the milk is breast milk, e.g., human breast milk. In some embodiments, the antibody is an immunoglobulin, e.g., IgA, IgD, IgE, IgG and IgM. In some embodiments, the immunoglobulin is an IgA class antibody. In further embodiments, the IgA class antibody is an IgA1 or IgA2 antibody. In certain embodiments, the IgA class antibody is a secretory IgA (sIgA) antibody. In some embodiments, the infant is a premature infant.

### BRIEF DESCRIPTION OF THE DRAWINGS

FIGS. 1A-1E depict that IgA binding to the intestinal bacteria of premature infants is positively correlated to breastfeeding and negatively correlated to the development of NEC. FIG. 1 depicts that fecal samples were obtained from premature infants and antibody binding IgA to intestinal bacteria was determined by flow cytometry. FIG. 2B depicts percent IgA bound bacteria from Breast fed (n=31) vs. formula fed (n=15) infants. The box represents the number of samples with <1% IgA binding of intestinal bacteria, Mann-Whitney test. FIG. 1C depicts that percent IgA binding was correlated with time post-delivery in breast fed and formula fed infants. The box represents the first month of life, Pearson's correlation coefficient. FIG. 1D depicts percent IgA bound bacteria from controls (n=13) or children diagnosed with NEC (n=18), from samples collected <days of life (DOL) 30; Mann-Whitney test. FIG. 1E depicts percent IgA bound intestinal bacteria from prospectively collected samples of NEC patients (n=6) and Controls (n=7), Pearson's correlation coefficient.

FIGS. 2A-2E depict that the IgA unbound fraction of the microbiota becomes dominated by a single bacterial taxon in the days preceding the development of NEC. Prospectively collected fecal samples were separated via magnetic separation into IgA positive and negative pools prior to targeted 16S ribosomal RNA (rRNA) sequencing and analysis of relative bacterial abundance. All samples analyzed prior to disease onset. FIG. 2A depicts mean relative abundance of different taxa between unsorted fecal samples from NEC patients and controls. FIG. 2B depicts Pielou evenness and FIG. 2C depicts Shannon diversity scores of IgA positive and IgA negative samples between NEC and controls grouped by day of life. *p>0.05; Kruskal-Wallis Test for multiple comparisons. FIG. 2D depicts relative abundances of NEC vs. control samples from the IgA negative fraction of the intestinal microbiota at an early (DOL 1-22), and late (DOL>22) age range compared by linear discriminant analysis effect size (LefSE) . FIG. 2E depicts mean relative abundance of different taxa between NEC patients and controls of IgA-positive and IgA-negative samples grouped by day of life. All NEC patients in this cohort develop NEC after day of life (DOL) 22.

FIGS. 3A-3F depict that IgA is necessary in breast milk to prevent the development of experimental NEC. FIG. 3A depicts experimental NEC mouse model. Wild-type pups are fed by dams that either can (C57BL/6) or cannot (Rag1^{-/-} or Igha^{-/-}) produce IgA. Formula fed mice are used as a positive control. FIG. 3B depicts IgA staining of the fecal matter of pups from FIG. 3A and shows absence of IgA bound bacteria in the Rag1−/− and Igha−/− dam breastfed pups and formula fed pups.

FIG. 3C depicts representative images of hematoxylin and eosin (H & E) staining of small intestine of pups from FIG. 3A. FIG. 3D depicts histology scores of the small intestines of pups from FIG. 3C. FIG. 3E depicts survival curve of pups from FIG. 3A, statistics determined by Log-rank (Mantel-Cox) test. FIG. 3F depicts weights of pups from FIG. 3A, statistical difference calculated by t-test on weights at experimental completion point. Data shown is grouped together from 3 individual experiments with minimum n=5 pups in each group and each experiment. *p<0.05, **p<0.01, ***p<0.001, and ****p<0.0001.

FIGS. 4A-4C depict that intestinal bacteria in premature infants is predominantly bound by maternal IgA. FIGS. 4A and 4B depict representative flow plots showing minimal binding of intestinal bacteria by IgM (FIG. 4A) and IgG (FIG. 4B) amongst premature infants. Numbers in quadrants show the mean percent+/−SD. FIG. 4C depicts percent IgA staining of intestinal bacteria of a premature infant fed with formula.

FIGS. 5A and 5B depict percent IgA-bound bacteria from longitudinally collected fecal samples from premature infants in the study. Dotted red line indicates the date of NEC diagnosis.

FIGS. 6A-6C depict magnetic separation and 16S rRNA gene sequencing of IgA positive and negative fraction of intestinal bacteria from premature infants. FIG. 6A depicts possible mechanisms for the drop in IgA binding that precedes the development of NEC. FIG. 6B depicts enrichment of the IgA bound fraction (IgA positive) and unbound (IgA negative) measured by flow cytometry after magnetic based sorting. FIG. 6C depicts mean relative abundance of various taxa grouped by disease incidence (NEC vs. CTRL) and IgA binding (positive vs. negative).

FIGS. 7A-7D depict relative frequencies of abundant OTUs from Unsorted, IgA positive and IgA negative fractions of the premature intestinal microbiota OTU abundances at the family level for the most highly represented taxa from the cohort in the Unsorted, IgA positive and IgA negative fractions additionally separated by day of life (Early=1-21; Late=22+).

FIG. 8 depicts relative abundance of different taxa of two premature infants who developed NEC at <14 days of life. Sample taken 1-2 days prior to diagnosis.

FIG. 9 depicts qPCR analysis of relative bacterial abundance from prospective premature infant fecal samples. Numbers based on 16S rRNA primers compared against a known number of E. coli analyzed using the same primers.

FIG. 10 depicts that Enterobacter spp. is enriched in the IgA positive fraction of mice without vaccination. Magnetically sorted IgA+/IgA− from fecal samples of d12 (d5 of NEC protocol) pups. qPCR for Enterobacter spp. (23S rRNA) as a fraction of the relative number of bacteria in each sample (as measured by 16S rRNA qPCR) *p-value=0.0379 by Paired t-test.

FIG. 11 depicts a bacterial array for determining the IgA repertoire of breast milk.

FIG. 12 depicts flow cytometry analyses of the specificity of a IgA repertoire of breast milk to intestinal bacterial strains.

FIG. 13 depicts a heatmap showing a fingerprint of the anti-bacterial repertoire of each individual mother.

FIG. 14 depicts intestinal bacteria of preterm infants is bound by IgA, but not by IgG or IgM.

FIG. 15 depicts percentage of IgA⁺ intestinal bacteria from preterm infants fed maternal milk or formula. Pearson correlation.

FIG. 16 depicts flow analysis of IgA bound to intestinal bacteria from maternal milk-fed preterm infants. Each color represents a different infant. Pearson correlation.

FIG. 17 depicts the relative abundance of Enterobacteriaceae in unsorted preterm fecal samples compared to percent IgA bound bacteria.

FIG. 18 depicts the ratio of reads (IgA⁻/IgA⁺; log2 trans.) from paired IgA positive and negative samples, graphed against DOL. Total bacterial reads (left), Enterobacteriaceae reads (right). Each color represents a different infant. Pearson correlation.

FIG. 19 depicts the scheme of the bacterial array and bacteria stained with maternal milk-derived IgA, co-stained with anti-human IgA and analyzed by flow cytometry. Shown are the percentages of IgA⁺ and mean fluorescence intensity (MFI) or IgA⁺ bacteria.

FIG. 20 depicts IgA from maternal milk donors analyzed as described in FIG. 19.

FIGS. 21A and 21B depict the breeding scheme (FIG. 21A) and flow cytometric analysis of IgA binding to mouse pup intestinal bacteria (FIG. 21B). Gated on SytoBC+. The numbers represent percentage in IgA⁺ quadrant.

FIGS. 22A and 22B depict histology (H&E) of mouse pups fed by different modalities in the murine NEC protocol (FIG. 22A) and survival of pups (FIG. 22B). Log-rank Mantel-Cox Test.

### DETAILED DESCRIPTION OF THE INVENTION

The presently disclosed subject matter relates to antibodies that bind to bacteria associated with NEC, methods of detecting the same, and methods of using the same for treating and/or preventing NEC. It is based, at least in part, on the discovery that maternal immunoglobulin A (IgA) is an important factor in protection against NEC, and a lack of IgA binding and domination of the intestinal microbiota can contribute to the development of disease.

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition to improving immunity, digestive function and/or decreasing inflammation, an effective amount of a composition described herein is an amount sufficient to improving immunity, digestive function and/or decreasing inflammation, as well as decrease the symptoms and/or reduce the likelihood of a digestive disorder and/or inflammation. An effective treatment described herein is a treatment sufficient to improving immunity, digestive function and/or decreasing inflammation, as well as decrease the symptoms and/or reduce the likelihood of a digestive disorder and/or inflammation. The decrease can be an about 1%, about 5%, about 10%, about 20%, about 30%, about 40%, about 50%, about 60%, about 70%, about 80%, about 90%, about 95%, about 98%, or about 99% decrease in severity of symptoms of a digestive disorder or inflammation, or the likelihood of a digestive disorder or inflammation. An effective amount can be administered in one or more administrations. A likelihood of an effective treatment described herein is a probability of a treatment being effective, i.e., sufficient to treat or ameliorate a digestive disorder and/or inflammation, as well as decrease the symptoms.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. The decrease can be an about 1%, about 5%, about 10%, about 20%, about 30%, about 40%, about 50%, about 60%, about 70%, about 80%, about 90%, about 95%, about 98%, or about 99% decrease in severity of complications or symptoms. “Treatment” can also mean prolonging survival as compared to expected survival if not receiving treatment.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

### 2. Intestinal Bacteria and Antibodies Binding to the Same

The presently disclosed subject matter is based, at least in part, on the discovery that maternal antibodies in breast milk bind to intestinal microbiota, and a lack of antibody binding of the intestinal microbiota can contribute to the development of NEC. As disclosed herein, a bacterium of the intestinal microbiota can be a bacterium associated with NEC.

In certain embodiments, the intestinal microbiota comprises bacteria of one or more family selected from the group consisting of Enterobacteriaceae, Pasteurellaceae, Pseudomonadaceae, Tissierellaceae, Veillonellaceae, Peptostreptococcaceae, Lachnospiraceae, Clostridiaceae, Enterococcaceae, Staphylococcaceae, Streptococcoceae and Bifidobacteriaceae.

In certain embodiments, the intestinal microbiota comprises bacteria of one or more genus selected from the group consisting of Enterobacter, Escherichia, Citrobacter, Salmonella, Klebsiella, Corynebacterium, Lactobacillus, Proteus, Haemophilus, Staphylococcus, Pseudomonas, Clostridium, Bifidobacterium, Enterococcus, Streptococcus and Veillonella.

In certain embodiments, the intestinal microbiota comprises one or more bacterium selected from the group consisting of E. coli NIHMB, E. coli AIEC 2A, E. coli CUMT8, Enterobacter, Enterococcus, E. coli NIHT5, Citrobacter, Klebsiella, Pseudomonas, Streptococcus, Yersinis, S. aureus (ptnA), Salmonella, S. aureus, Enterobacter (NECteria monoculture), Escherichia (ECORL), Escherichia (ECMB), Escherichia (ECT5), Escherichia (MT8), Escherichia coli 909, Escherichia coli 910, Escherichia coli 4185, Citrobacter rodentium 51459, Salmonella typhimurium 3261, Klebsiella pneumoniae, Enterobacter cloacae, Klebsiella oxytoca, Klebsiella aerogenes, Corynebacterium spp., Lactobacillus casei, Lactobacillus paracasei, Proteus mirabilis, Streptococcus agalactiae, Streptococcus salivarius, Streptococcus pneumoniae 19F, Veillonella parvula, Veillonella atypica, Enterococcus faecalis 19433, Enterococcus faecalis 286, Enterococcus faecium, Bifidobacterium breve, Bifidobacterium bifidum, Clostridium butyricum, Clostridium sporogenes, Clostridium perfringens, Pseudomonas aerginosa 01, Staphylococcus aureus, Staph aureus Protein A-, MSSA NARSA227, MSSA NARSA235, Staphylococcus capitis, Staph epidermidis B1468, Staph epi. NARSA101, Staph epi. NIHLM087, Staph saprophyticus, Haemophilus parainfluenza, and S. epidermidis.

In certain embodiments, intestinal bacteria associated with NEC comprises Enterobacteriaceae, Streptococcoceae, Veillonellaceae, Enterococcaceae, Bifidobacteriaceae, Clostridiaceae, Pseudomonadaceae, Staphylococcaceae and/or Pasteurellaceae. In certain embodiments, intestinal bacteria associated with NEC comprises Enterobacter, Escherichia, Citrobacter, Salmonella, Klebsiella, Corynebacterium, Lactobacillus, Proteus, Haemophilus, Staphylococcus, Pseudomonas, Clostridium, Bifidobacterium, Enterococcus, Streptococcus and/or Veillonella. In certain embodiments, intestinal bacteria associated with NEC comprises Enterobacter (NECteria monoculture), Escherichia (ECORL), Escherichia (ECMB), Escherichia (ECTS), Escherichia (MT8), Escherichia coli 909, Escherichia coli 910, Escherichia coli 4185, Citrobacter rodentium 51459, Salmonella typhimurium 3261, Klebsiella pneumoniae, Enterobacter cloacae, Klebsiella oxytoca, Klebsiella aerogenes, Corynebacterium spp., Lactobacillus casei, Lactobacillus paracasei, Proteus mirabilis, Streptococcus agalactiae, Streptococcus salivarius, Streptococcus pneumoniae 19F, Veillonella parvula, Veillonella atypica, Enterococcus faecalis 19433, Enterococcus faecalis 286, Enterococcus faecium, Bifidobacterium breve, Bifidobacterium bifidum, Clostridium butyricum, Clostridium sporogenes, Clostridium perfringens, Pseudomonas aerginosa 01, Staphylococcus aureus, Staph aureus Protein A-, MSSA NARSA227, MSSA NARSA235, Staphylococcus capitis, Staph epidermidis B1468, Staph epi. NARSA101, Staph epi. NIHLM087, Staph saprophyticus and/or Haemophilus parainfluenza. In further embodiments, the intestinal bacteria associated with NEC comprises C. rodentium, E. aerogenes, E. cloacae, E. coli 587, E. coli 596, E. coli 605, E. coli 909, E. coli 910, E. coli 4185, E. coli ECO2A, E. coli ECMB, E. coli ECTS, E. coli MTB, S. typhimurium SL3261, Enterobacter NECMONO, K. aerogenes 13048, K. oxytoca 43165, K. oxytoca K405, K. pneumoniae, S. marcescens 855, S. marcescens 853, P. mirabilis, P. vulgaris, P. aeruginosa, M. nonliquefaciens, L. casei, S. agalactiae, S. aureus RS, S. aureus TWH, S. capitis, S. epidermidis LM087, S. epidermidis LM088, S. epidermidis RS, S. saprophyticus, E. faecalis 2649, E. faecalis 19433, and E. faecium. In some embodiments, any of the families, genera, or species of any one of the preceding bacteria is associated with NEC.

An antibody that binds to any intestinal bacteria and/or bacteria associated with NEC disclosed herein can be a maternal antibody. In certain embodiments, the maternal antibody is an immunoglobulin. In certain embodiments, the maternal antibody comprises an IgA, IgD, IgE, IgG, and/or IgM. In certain embodiments, the maternal antibody comprises an IgA. In certain embodiments, the maternal antibody has a K_{d} of at most about 10⁻⁶ M, about 10⁻⁷ M, about 10⁻⁸M, about 10⁻⁹M, about 10⁻¹⁰ M, about 10⁻¹¹ M, about 10⁻¹²M or less.

In certain embodiments, a maternal antibody disclosed herein is comprised in breast milk. In certain embodiments, the breast milk is human breast milk. In certain embodiments, the breast milk is stored in a breast milk bank. In certain embodiments, the breast milk is produced by a mother of a premature infant. In certain embodiments, the maternal antibody is present in breast milk at a concentration of at least about 0.000001% w/w, at least about 0.00001% w/w, at least about 0.0001% w/w, at least about 0.001% w/w, at least about 0.01% w/w, at least about 0.1% w/w, at least about 1% w/w or more of the breast milk. In certain embodiments, the maternal antibody is present in breast milk at a concentration of at least about 0.01 ppm, at least about 0.1 ppm, at least about 1 ppm, at least about 10 ppm, at least about 100 ppm, at least about 1000 ppm or more of the breast milk.

3. Methods of Detection

The presently disclosed subject matter provides a method of detecting an antibody in a sample. In certain embodiments, the method comprises: (a) providing a a bacterial array; (b) contacting the bacterial array with the sample; and (c) detecting the antibody by detecting the binding of the antibody to a bacterium of the bacterial array. In certain embodiments, one or more antibody, e.g., an antibody repertoire, is isolated from the sample before contacting the bacterial array. In certain embodiments, the method further comprises washing the bacterial array before detecting the binding of an antibody to the bacterial array.

In certain embodiments, the breast milk is human breast milk. In certain embodiments, the breast milk is stored in a breast milk bank. In certain embodiments, the breast milk is produced by a mother of a premature infant.

In certain embodiments, the bacterial array comprises any bacterium associated with NEC or any intestinal bacteria disclosed herein. For example, the bacterial array can include bacteria of one or more, two or more, three or more, four or more, five or more, six or more, seven or more, eight or more, nine or more, ten or more, or eleven or more of the families selected from the group consisting of Enterobacteriaceae, Pasteurellaceae, Pseudomonadaceae, Tissierellaceae, Veillonellaceae, Peptostreptococcaceae, Lachnospiraceae, Clostridiaceae, Enterococcaceae, Staphylococcaceae, Streptococcoceae and Bifidobacteriaceae. In particular examples, the bacterial array includes bacteria of the families Enterobacteriaceae, Pasteurellaceae, Pseudomonadaceae, Tissierellaceae, Veillonellaceae, Peptostreptococcaceae, Lachnospiraceae, Clostridiaceae, Enterococcaceae, Staphylococcaceae, Streptococcoceae and Bifidobacteriaceae. For example, the bacterial array includes bacteria of the Enterobacteriaceae family.

In further examples, the bacterial array includes bacteria of one or more, two or more, three or more, four or more, five or more, six or more, seven or more, eight or more, nine or more, ten or more, eleven or more, twelve or more, thirteen or more, fourteen or more, or fifteen or more of the genera selected from the group consisting of Enterobacter, Escherichia, Citrobacter, Salmonella, Klebsiella, Corynebacterium, Lactobacillus, Proteus, Haemophilus, Staphylococcus, Pseudomonas, Clostridium, Bifidobacterium, Enterococcus, Streptococcus and Veillonella. In particular examples, the bacterial array comprises bacteria of the genera Enterobacter, Escherichia, Citrobacter, Salmonella, Klebsiella, Corynebacterium, Lactobacillus, Proteus, Haemophilus, Staphylococcus, Pseudomonas, Clostridium, Bifidobacterium, Enterococcus, Streptococcus and Veillonella.

In still further examples, the bacterial array includes at least one of the bacteria selected from the group consisting of E. coli NIHMB, E. coli AIEC 2A, E. coli CUMT8, Enterobacter, Enterococcus, E. coli NIHT5, Citrobacter, Klebsiella, Pseudomonas, Streptococcus, Yersinis, S. aureus (ptnA), Salmonella, S. aureus, Enterobacter (NECteria monoculture), Escherichia (ECORL), Escherichia (ECMB), Escherichia (ECT5), Escherichia (MT8), Escherichia coli 909, Escherichia coli 910, Escherichia coli 4185, Citrobacter rodentium 51459, Salmonella typhimurium 3261, Klebsiella pneumoniae, Enterobacter cloacae, Klebsiella oxytoca, Klebsiella aerogenes, Corynebacterium spp., Lactobacillus casei, Lactobacillus paracasei, Proteus mirabilis, Streptococcus agalactiae, Streptococcus salivarius, Streptococcus pneumoniae 19F, Veillonella parvula, Veillonella atypica, Enterococcus faecalis 19433, Enterococcus faecalis 286, Enterococcus faecium, Bifidobacterium breve, Bifidobacterium bifidum, Clostridium butyricum, Clostridium sporogenes, Clostridium perfringens, Pseudomonas aerginosa 01, Staphylococcus aureus, Staph aureus Protein A-, MSSA NARSA227, MSSA NARSA235, Staphylococcus capitis, Staph epidermidis B1468, Staph epi. NARSA 101, Staph epi. NIHLM087, Staph saprophyticus, Haemophilus parainfluenza, and S. epidermidis. In particular examples, the bacterial array includes E. coli NIHMB, E. coli AIEC 2A, E. coli CUMT8, Enterobacter, Enterococcus, E. coli NIHT5, Citrobacter, Klebsiella, Pseudomonas, Streptococcus, Yersinis, S. aureus (ptnA-), Salmonella, S. aureus, Enterobacter (NECteria monoculture), Escherichia (ECORL), Escherichia (ECMB), Escherichia (ECTS), Escherichia (MT8), Escherichia coli 909, Escherichia coli 910, Escherichia coli 4185, Citrobacter rodentium 51459, Salmonella typhimurium 3261, Klebsiella pneumoniae, Enterobacter cloacae, Klebsiella oxytoca, Klebsiella aerogenes, Corynebacterium spp., Lactobacillus casei, Lactobacillus paracasei, Proteus mirabilis, Streptococcus agalactiae, Streptococcus salivarius, Streptococcus pneumoniae 19F, Veillonella parvula, Veillonella atypica, Enterococcus faecalis 19433, Enterococcus faecalis 286, Enterococcus faecium, Bifidobacterium breve, Bifidobacterium bifidum, Clostridium butyricum, Clostridium sporogenes, Clostridium perfringens, Pseudomonas aerginosa 01, Staphylococcus aureus, Staph aureus Protein A-, MSSA NARSA227, MSSA NARSA235, Staphylococcus capitis, Staph epidermidis B1468, Staph epi. NARSA101, Staph epi. NIHLM087, Staph saprophyticus, Haemophilus parainfluenza, and S. epidermidis.

In yet further examples, the bacterial array includes at least one bacterium selected from the group consisting of C. rodentium, E. aerogenes, E. cloacae, E. coli 587, E. coli 596, E. coli 605, E. coli 909, E. coli 910, E. coli 4185, E. coli ECO2A, E. coli ECMB, E. coli ECTS, E. coli MTB, S. typhimurium SL3261, Enterobacter NECMONO, K. aerogenes 13048, K. oxytoca 43165, K. oxytoca K405, K. pneumoniae, S. marcescens 855, S. marcescens 853, P. mirabilis, P. vulgaris, P. aeruginosa, M. nonliquefaciens, L. casei, S. agalactiae, S. aureus RS, S. aureus TWH, S. capitis, S. epidermidis LM087, S. epidermidis LM088, S. epidermidis RS, S. saprophyticus, E. faecalis 2649, E. faecalis 19433, and E. faecium. In particular examples, the bacterial array comprises C. rodentium, E. aerogenes, E. cloacae, E. coli 587, E. coli 596, E. coli 605, E. coli 909, E. coli 910, E. coli 4185, E. coli ECO2A, E. coli ECMB, E. coli ECTS, E. coli MTB, S. typhimurium SL3261, Enterobacter NECMONO, K. aerogenes 13048, K. oxytoca 43165, K. oxytoca K405, K. pneumoniae, S. marcescens 855, S. marcescens 853, P. mirabilis, P. vulgaris, P. aeruginosa, M nonliquefaciens, L. casei, S. agalactiae, S. aureus RS, S. aureus TWH, S. capitis, S. epidermidis LM087, S. epidermidis LM088, S. epidermidis RS, S. saprophyticus, E. faecalis 2649, E. faecalis 19433, and E. faecium.

In certain embodiments, the bacterial array comprises one or more intestinal bacteria associated with NEC. In certain embodiments, the bacterial array comprises a substrate to which intestinal bacteria are affixed. In certain embodiments, the substrate is transparent or otherwise suitable for detecting a detectable label. In certain embodiments, the substrate is a multiwell plate. In certain embodiments, the bacterial array comprises at least about 1, at least about 2, at least about 3, at least about 4, at least about 5, at least about 10, at least about 15, at least about 20 or more bacteria genera associated with NEC. In certain embodiments, the bacterial array comprises at least about 1, at least about 2, at least about 3, at least about 4, at least about 5, at least about 10, at least about 15, at least about 20 or more bacteria families associated with NEC.

In certain embodiments, the antibody is an immunoglobulin. In certain embodiments, the antibody is an IgA, IgD, IgE, IgG, or IgM. Methods for detecting and/or determining the level of an antibody, e.g., an immunoglobulin, are well known to those skilled in the art, and include, but are not limited to, flow cytometry, mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation, and immunohistochemistry. These methods use antibodies, or antibody equivalents, to detect protein, or use biophysical techniques. In certain embodiments, an immunoglobulin is detected by an antibody that binds to the constant region of the immunoglobulin. In certain embodiments, an immunoglobulin is detected by an antibody that binds to the Fc region of the immunoglobulin. The term “Fc region” is used to define a C-terminal region of an immunoglobulin heavy chain. In certain embodiments, the Fc region possesses an effector function.

In certain embodiments, the antibody is an immunoglobulin, e.g., IgA, IgD, IgE, IgG and IgM. In certain embodiments, the immunoglobulin is detected by an anti-immunoglobulin antibody. In certain embodiments, the anti-immunoglobulin antibody comprises a detectable label. In certain embodiments, the binding of the antibody to the bacterial array is detected by flow cytometry. In certain embodiments, the antibody is an IgA, e.g., an IgAl or IgA2 antibody. For example, the IgA antibody can be a secretory IgA (sIgA) antibody. In certain embodiments, the IgA class antibody is detected by an anti-IgA antibody. In certain embodiments, the anti-IgA antibody comprises a detectable label.

In certain embodiments, a detection method for measuring an immunoglobulin includes the steps of: contacting an immunoglobulin, or a biological sample comprising the same, with an antibody or variant (e.g., fragment) thereof, which selectively binds the immunoglobulin, and detecting whether the antibody or variant thereof is bound to the immunoglobulin. The antibody can comprise a detectable label. The method can further include contacting the sample with a second antibody, e.g., a labeled antibody. The method can further include one or more washing steps, e.g., to remove one or more reagents.

Labeled antibodies against an immunoglobulin can be used for detection purposes. Suitable detectable labels include radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as fluorescein, rhodamine, phycoerythrin (PE), allophycocyanin (APC), and biotin. Immunoenzymatic interactions can be visualized using different enzymes such as peroxidase, alkaline phosphatase, or different chromogens such as 3,3′-diaminobenzidine (DAB), 3-amino-9-ethylcarbazole (AEC), or Fast Red. The labeled antibody or antibody fragment will preferentially accumulate at the location of bacteria which are bound by the immunoglobulin. The labeled antibody or variant thereof, e.g., antibody fragment, can then be detected using known techniques, such as flow cytometry.

Antibodies of the present disclosure include any antibody, whether natural or synthetic, full length or a fragment thereof, monoclonal or polyclonal, that binds sufficiently strongly and specifically to the biomarker to be detected. An antibody can have a dissociation constant (K_{d}) of at most about 10⁻⁶ M, about 10⁻⁷ M, about 10⁻⁸ M, about 10⁻⁹ M, about 10⁻¹⁰ M, about 10⁻¹¹ M, about 10⁻¹² M or less. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies, and derivatives thereof, that can be used in the context of the present disclosure encompass polyclonal or monoclonal antibodies, synthetic and engineered antibodies, chimeric, human, humanized, or single-chain antibodies, phase produced antibodies (e.g., from phage display libraries), as well as functional binding fragments thereof. For example, antibody fragments capable of binding to an immunoglobulin, or portions thereof, including, but not limited to, Fv, Fab, Fab′ and F(ab′)2 fragments, can be used. Such fragments can be produced by enzymatic cleavage or by recombinant techniques.

In certain embodiments, the method further comprises selecting the breast milk for administering to an infant, when antibodies that binds to bacteria associated with necrotizing enterocolitis (NEC) are detected.

### 4. Methods of Treatment

The presently disclosed subject matter provides a method of treating, preventing, and/or reducing at least one symptom of NEC in an infant in need thereof. In certain embodiments, the method comprises: detecting an antibody in a sample of breast milk that binds to one or more intestinal bacteria disclosed herein. In certain embodiments, the method further comprises administering an effective amount of the breast milk to an infant, when an antibody that binds to one or more bacteria associated with necrotizing enterocolitis (NEC) are detected.

In certain embodiments, the method comprises: administering an effective amount of a composition to the infant, wherein the composition comprises an effective amount of antibodies that bind to one or more intestinal bacteria disclosed herein.

In certain embodiments, the composition is milk or an infant formula. In certain embodiments, the intestinal bacteria are associated with NEC.

In certain embodiments, the infant is a premature infant. In certain embodiments, the infant is born at less than about 37 weeks gestation, less than about 36 weeks gestation, less than about 35 weeks gestation, less than about 34 weeks gestation, less than about 33 weeks gestation, less than about 32 weeks gestation, less than about 31 weeks gestation, less than about 30 weeks gestation, less than about 29 weeks gestation, less than about 28 weeks gestation, less than about 27 weeks gestation, less than about 26 weeks gestation, less than about 25 weeks gestation, less than about 24 weeks gestation, less than about 23 weeks gestation, or less than about 22 weeks gestation. In certain embodiments, the infant is at risk of developing NEC. In certain embodiments, the infant is less than about 1 year old, less than about 11 months old, less than about 10 months old, less than about 9 months old, less than about 8 months old, less than about 7 months old, less than about 6 months old, less than about 5 months old, less than about 4 months old, less than about 3 months old, less than about 2 months old, or less than about 1 month old. In certain embodiments, the infant is less than about 10 weeks old, less than about 9 weeks old, less than about 8 weeks old, less than about 7 weeks old, less than about 6 weeks old, less than about 5 weeks old, less than about 4 weeks old, less than about 3 weeks old, less than about 2 weeks old, or less than about 1 week old. In certain embodiments, the infant is less than about 10 days old, less than about 9 days old, less than about 8 days old, less than about 7 days old, less than about 6 days old, less than about 5 days old, less than about 4 days old, less than about 3 days old, less than about 2 days old, or less than about 1 day old.

In certain embodiments, the infant is between about 1 day old and about 10 weeks old, between about 2 day old and about 9 weeks old, between about 3 day old and about 8 weeks old, between about 4 day old and about 7 weeks old, between about 4 day old and about 6 weeks old, between about 1 day old and about 5 weeks old, between about 1 day old and about 4 weeks old, between about 1 day old and about 3 weeks old, between about 1 day old and about 2 weeks old, or between about 1 day old and about 1 week old. In certain embodiments, the infant is between about 1 month old and about 1 year old, between about 1 year old and about 2 years old, between about 2 years old and about 3 years old, between about 3 years old and about 4 years old, between about 4 years old and about 5 years old, between about 5 years old and about 10 years old, or between about 10 years old and about 15 years old.

In certain embodiments, the infant is more than about 1 day old, more than about 2 days old, more than about 3 days old, more than about 4 days old, more than about 5 days old, more than about 6 days old, more than about 1 week old, more than about 2 weeks old, more than about 3 weeks old, more than about 4 weeks old, more than about 1 month old, more than about 2 months old,. more than about 3 months old, more than about 4 months old, more than about 5 months old, more than about 6 months old, more than about 1 year old, more than about 2 years old, more than about 3 years old, more than about 4 years old, or more than about 5 years old.

In certain embodiments, the composition can be fed to an infant from 20 times per day to once per day, from 10 times per day to once per day, or from 5 times per day to once per day. In certain embodiments, the composition can be fed to an infant once per day, twice per day, thrice per day, 4 times per day, 5 times per day, 6 times per day, 7 times per day, 8 times per day, 9 times per day, 10 or more times per day. In certain embodiments, the composition can be fed to an infant once per two days, once per three days, once per four days, once per five days, once per six days, once a week, once per two weeks, once per three weeks, or once per month. In certain embodiments, the composition can be fed to an infant in a constant manner.

In certain embodiments, the dosage of the antibody is between about 1 mg/kg body weight per day and about 5000 mg/kg body weight per day. In certain embodiments, the dosage of the antibody is between about 5 mg/kg body weight per day and about 1000 mg/kg body weight per day, between about 10 mg/kg body weight per day and about 500 mg/kg body weight per day, between about 10 mg/kg body weight per day and about 250 mg/kg body weight per day, between about 10 mg/kg body weight per day and about 200 mg/kg body weight per day, between about 20 mg/kg body weight per day and about 100 mg/kg body weight per day, between about 20 mg/kg body weight per day and about 50 mg/kg body weight per day or any intermediate range thereof. In certain embodiments, the dosage of the antibody is at least about 1 mg/kg body weight per day, at least about 5 mg/kg body weight per day, at least about 10 mg/kg body weight per day, at least about 20 mg/kg body weight per day, at least about 50 mg/kg body weight per day, at least about 100 mg/kg body weight per day, at least about 200 mg/kg body weight per day or more. In certain embodiments, the dosage of the antibody is no more than about 5 mg/kg body weight per day, no more than about 10 mg/kg body weight per day, no more than about 20 mg/kg body weight per day, no more than about 50 mg/kg body weight per day, no more than about 100 mg/kg body weight per day, no more than about 200 mg/kg body weight per day, no more than about 500 mg/kg body weight per day or more.

In certain embodiments, the concentration of at least one antibody decreases over the course of the treatment. In certain embodiments, the concentration of at least one antibody increases over the course of the treatment. In certain embodiments, the concentration of at least one antibody is modified based on the age of the infant.

In certain embodiments, the composition comprises antibodies that bind to at least about 1, at least about 2, at least about 3, at least about 4, at least about 5, at least about 10, at least about 15, at least about 20, at least about 30, at least about 40, at least about 50 or more bacterial species associated with NEC. In certain embodiments, the composition comprises antibodies that bind to at least about 1, at least about 2, at least about 3, at least about 4, at least about 5, at least about 10, at least about 15, at least about 20, at least about 30, at least about 40, at least about 50 or more bacterial genera associated with NEC. In certain embodiments, the composition comprises antibodies that bind to at least about 1, at least about 2, at least about 3, at least about 4, at least about 5, at least about 10, at least about 15, at least about 20, at least about 30, at least about 40, at least about 50 or more bacterial families associated with NEC.

In certain embodiments, the composition can further comprise an additional agent that is beneficial to an infant's health and/or treatment of NEC.

### EXAMPLES

The presently disclosed subject matter will be better understood by reference to the following examples, which is provided as exemplary of the invention, and not by way of limitation.

### Example 1

### Introduction

This Example shows that maternal immunoglobulin A (IgA) is an important factor in protection against NEC. Analysis of IgA-binding on fecal samples from premature infants indicated that breast milk was the predominant source of IgA in the first month of life, and that a relative drop in the fraction of bacteria bound by IgA was associated with the development of NEC. Sequencing of IgA-bound and unbound bacteria indicated that NEC was associated with a unique decrease in the diversity of IgA unbound bacteria which indicated that a lack of IgA binding and domination of the microbiota by specific taxa, can contribute to the development of disease. Further, it was confirmed that IgA was critical in preventing NEC in a murine model, where pups reared by IgA deficient mothers are susceptible to the disease. This study illustrates the importance of maternal IgA in shaping the host-microbiota relationship of neonates and provides evidence that IgA is a necessary and critical factor in breast milk for the prevention of NEC.

### Materials and Methods

### Mice

C57BL/6 mice were purchased from Taconic Biosciences, Inc. Rag1^{-/-} mice were obtained from The Jackson Laboratory. Igha^{-/-} mice were obtained from Dr. Yasmine Belkaid (NIH/NIAID). All mice were maintained at and all experiments were performed in an American Association for the

Accreditation of Laboratory Animal Care-accredited animal facility at the University of Pittsburgh and housed in accordance with the procedures outlined in the Guide for the Care and Use of Laboratory Animals under an animal study proposal approved by the Institutional Animal Care and Use Committee of the University of Pittsburgh. Mice were housed in specific pathogen-free (SPF) conditions.

### Human Fecal Samples

The human study protocol was approved by the Institutional Review Board (Protocol Nos. PRO16030078, PRO09110437) of the University of Pittsburgh. Fecal samples were collected fresh or from the diaper of preterm infants at Magee-Womens Hospital of UPMC, Pittsburgh and frozen immediately at −80° C. The samples were later divided into age-matched controls and NEC depending on the incidence of NEC.

### Fecal IgA Flow Cytometry and Magnetic Sorting of IgA+ and IgA− Bacteria

Either fecal pellets collected from mice after sacrifice or ˜50 mg of frozen human fecal material was placed in 1.5 mL Eppendorf tubes and 1 mL Phosphate Buffered Saline (PBS) was added. The fecal material was disrupted by a combination of vortexing and pipetting and passed through a 40 μm filter to remove food or fibrous material. The fecal material was diluted with PBS to obtain a bacterial optical density (OD) of ˜0.4 to maintain equality between samples and to prevent the magnetic columns from clogging. A volume of 200 μL of the suspended bacterial material was then frozen as an “unsorted” control. An additional 200 μL of the suspended material was divided equally on a 96-well plate for IgA staining and isotype control for each sample to eliminate non-specific binding. The fractions were washed with twice with staining buffer (1% Bovine Serum Albumin (Sigma) in PBS-filtered through a 2.2 μm filter). The bacteria were with Syto BC (Green Fluorescent nuclear acid stain, Invitrogen-1:400), APC Anti-Human IgA (Miltenyi Biotec clone IS11-8E10) (1:10), Anti-Human IgM BV421 (BD Biosciences clone G20-127) (1:30)/ BV421 Mouse Anti-Human IgG (BD Horizon clone X40) (1:30) or PE-conjugated Anti-Mouse IgA (eBioscience clone mA-6E1) (1:500), Anti-Mouse Rat IgM BV421 (BD Horizon clone R6-60.2) (1:30) or Anti-Mouse Rat IgG2a isotype (BD Horizon clone R35-95) and blocking buffer of 20% Normal Mouse Serum for human or 20% Normal Rat Serum for mouse samples (ThermoFisher). The isotype control was stained similarly using APC Mouse IgG1 isotype control (Miltenyi Biotec clone-IS5-21F5) (1:10) or PE-conjugated Rat Anti-Mouse IFNγ (eBioscience clone XMG1.2). The stained samples were incubated in dark for an hour at 4° C. Samples were then washed three times with 200 μL of staining buffer before flow-cytometric analysis (LSRFortessa-BD Biosciences).

For magnetic activated cell sorting (MACS), 500 μL of the suspended fecal material was used to compensate for the loss of material during sorting and scaled the staining volume accordingly. Anti-IgA stained fecal bacterial pellets were incubated in 1 mL per sample of staining buffer containing 45 μL of anti-APC or anti-PE MACS Microbeads (Miltenyi Biotec) (20 min at 4° C. in dark), washed twice with 1 mL Staining Buffer (8000×rpm, 5 min, 4° C.), and then sorted using MS columns (Miltenyi Biotec). The flow-through was collected as IgA-unbound (IgA-negative) fraction. Columns were washed with 70% ethanol and sterile PBS between separations and each sample was run five times to increase purity. The IgA-bound fraction was added in the column and the steps mentioned above were repeated four times for maximum enrichment. 100 μL each of the IgA-bound and IgA-unbound fraction was used for post-sort flow cytometric analysis (along with unsorted sample).

### DNA Extraction

All microbial DNA was extracted using the MO BIO PowerSoil DNA Isolation kit (single tube extractions). The unsorted, IgA-bound and IgA-unbound pellets were resuspended in Solution TD1 by pipetting and vortexing and ˜200 μL of 0.1 mm diameter Zirconia/Silica beads (Biospec) were added and shaken horizontally on a lab mixer for 12-18 min at maximum speed using a MO BIO vortex adaptor. All remaining steps followed the manufacturer's protocol. The DNA extracted was stored at −20° C. for further 16S amplicon PCR and sequencing.

### 16S Amplicon PCR, Sequencing and Analysis

PCR amplification of the small subunit ribosomal RNA (16S rRNA) gene was performed in triplicate 25 μL reactions. Reactions were held at 94° C. for 3 min to denature the DNA, with amplification performed for 30 cycles at 94° C. for 45 s, 50° C. for 60 s, and 72° C. for 90 s; followed by a final extension of 10 min at 72° C. Amplicons were produced utilizing primers adapted for the Illumina MiSeq. Amplicons target the V4 region and primers utilized either the Illumina adaptor, primer pad and linker (forward primer) or Illumina adaptor, Golay barcode, primer pad and linker (reverse primer) followed by a sequence targeting a conserved region of the bacterial 16S rRNA gene as described^{51,52}. The only deviation from the protocol was that PCR was run for 30 cycles. Amplicons were cleaned using the Qiagen UltraClean 96 PCR Cleanup Kit. Quantification of individual amplicons was performed with the Invitrogen Quant-iT dsDNA High Sensitivity Assay Kit. Amplicons were then pooled in equimolar ratio. Agarose gel purification was performed to further purify the amplicon pool and remove undesired PCR products prior to submission for paired-end sequencing on the Illumina MiSeq. Read pairing, clustering and core diversity statistics were generated through PEAR, UPARSE and QIIME and R^{53,54}. LEfSe was used to compare family level relative abundances between NEC and control groups at late and early time points, as well as within the same groups at different time points⁵⁵.

### Deconvolution and Microbiome Data Analysis:

Flow cytometry was used to determine the percentage of IgA positive and IgA negative bacteria in each sample (unsorted, IgA positive, IgA negative) post-separation. Raw reads were deconvoluted by creating a set of two linear equations per OTU with the known percentages from flow cytometry as follows:

f1x+f2y=z1

f3x+f4y=z2

The raw number of reads in an OTU in the sorted IgA positive and IgA negative samples are denoted by z1 and z2, respectively. The percentages of IgA positive and IgA negative bacteria in the sorted IgA positive sample are denoted by f1 and f2, respectively. The percentages of IgA positive and IgA negative bacteria in the sorted IgA negative sample are denoted by f3 and f4, respectively. The deconvoluted number of reads in the IgA positive and IgA negative samples are denoted by x and y and were solved by minimizing error. The deconvoluted data was processed through the QIIME2 workflow to create alpha diversity metrics with sampling depth chosen based on alpha rarefaction plotting.

Quantitative PCR for 16S rRNA.

PCR amplification of the small subunit ribosomal RNA (16S rRNA) gene was performed in triplicate 10 μL reactions. Reactions were held at 95° C. for 3 min to denature the DNA, with amplification performed for 35 cycles (95° C. for 10 s and 60° C. for 30 s). The forward primer sequence of 16S is ACTCCTACGGGAGGCAGCAGT and the reverse primer sequence of 16S ATTACCGCGGCTGCTGGC.

### Quantitative PCR for Enterobacter spp.

PCR amplification of the small subunit ribosomal RNA (23S rRNA) gene was performed in triplicate 10 μL reactions. Reactions were held at 95° C. for 3 min to denature the DNA, with amplification performed for 35 cycles (95° C. for 10 s and 60° C. for 30 s). The forward primer sequence of Enterobacter 23S is AGTGGAACGGTCTGGAAAGG and the reverse primer sequence of Enterobacter 23S TCGGTCAGTCAGGAGTATTTAGC⁵⁶.

### Induction of NEC

NEC was induced in 7- to 8-day-old mice (weighing <4g) by hand-feeding mice formula via gavage 5 times/day (22-gauge needle; 200 μL volume; Similac Advance infant formula [Ross Pediatrics, Columbus, Ohio]/Esbilac canine milk replacer 2:1). The formula is supplemented with 10⁷ CFUs of Enterobacter spp. (99%) and Enterococcus spp. (1%) and mice are rendered hypoxic (5% O2, 95% N2) for 10 minutes in a hypoxic chamber (Billups-Rothenberg, Del Mar, Calif.) twice daily for 4 days^{6,57}. Males and females were used in all experiments. Disease was monitored by weighing mice daily prior to the second feed. The severity of disease was determined on histologic sections of the entire length of the small intestines stained with hematoxylin and eosin by trained personnel who were blinded to the study conditions according to previously published scoring system from 0 (normal) to 4 (severe)⁵⁸.

### Statistics

Statistical tests used are indicated in the figure legends. Data are presented as mean. Group sizes were determined based on the results of preliminary experiments. Mouse studies were performed in a non-blinded fashion. Statistical significance was determined with the two-tailed unpaired Student's t-test or non-parametric Mann-Whitney test when comparing two groups and one-way ANOVA with multiple comparisons, when comparing multiple groups. All statistical analyses were calculated using Prism software (GraphPad). Differences were considered to be statistically significant when p<0.05.

TABLE 1

| | NEC | Control |
|---|---|---|
| No. of patients | 20 | 26 |
| Avg. gestational age | 29 5/7 | 29 1/7 |
| at birth (in weeks) | | |
| NEC at study day (Day of | 22.48 | NA |
| life [DOL]) (in days) | | |
| Sex | Females-6 | Females-5 |
| | Males-14 | Males-21 |
| Avg. weight (in grams) | 1291.28 | 1207.19 |
| Mode of Delivery | Vaginal-7 | Vaginal-5 |
| | C-section-13 | C-section-21 |
| Feeding | Breast-fed-11 | Breast-fed-20 |
| | Formula-fed-9 | Formula-fed-6 |

TABLE 2

| | NEC | Control |
|---|---|---|
| No. of patients | 6 | 7 |
| Avg. gestational age at | 27 0/7 | 26 6/7 |
| birth (in weeks) | | |
| NEC at study day (Day of | 23.33 | NA |
| life [DOL]) (in days) | | |
| Sex | Females-2 | Females-5 |
| | Males-4 | Males-2 |
| Avg. weight (in grams) | 944.33 | 927.14 |
| Mode of Delivery | Vaginal-1 | Vaginal-5 |
| | C-section-5 | C-section-2 |

### Results and Discussion

Necrotizing enterocolitis is a debilitating disease of preterm infants, affecting about 7% of very low birth weight infants and resulting in both high mortality (>20%) and lifelong complications amongst infants who recover^{1,2}. The exact etiology of NEC is unknown, but disease is believed to occur subsequent to intestinal epithelial damage, bacterial invasion, and immune-mediated inflammation³⁻⁵. The incidence of disease is significantly higher in infants fed with artificial formula relative to those receiving maternal or donor milk, but the mechanism(s) for this protective effect remain unknown⁶⁻⁹. NEC has been associated with shifts in the intestinal microbiota, most commonly an increased relative abundance of Enterobacteriaceae, but this increase is not sufficient for disease and lacks predictive power¹⁰⁻¹¹.

How breast milk affects the bacteria of the intestine has been a focus of investigation with milk oligosaccharides and anti-microbials being two of the most promising possibilities¹²⁻¹⁵. Breast milk also contains large amount of antibodies, primarily IgA, with smaller amounts of immunoglobulin M (IgM) and immunoglobulin G (IgG)¹⁶. IgA in particular has been shown to be important in shaping the development of the pediatric microbiota by promoting maturation of the community away from Proteobacteria, towards anaerobic Firmicutes and Bacteroidetes^{17,18}. Interestingly, the IgA-secreting B cells that provide breast milk IgA are derived from the small intestine, indicating that the IgA repertoire of breast milk is primarily targeted against intestinal bacteria and can be biased towards the most common organisms of the maternal microbiota^{16,19-21}. For various reasons, including exposure to antibiotics and an underdeveloped gastrointestinal tract, preterm infants harbor gut microbial communities that are distinct from those of healthy term infants and adults²². Moreover, the preterm gut is rich in the facultative anaerobes (Enterobacteriaceae, Staphylococcaceae) that are relatively rare in the maternal intestinal microbiota¹⁰. Therefore, it was hypothesized that IgA in breast milk can prevent the development of NEC by inhibiting bacteria from accessing and damaging the gut mucosa.

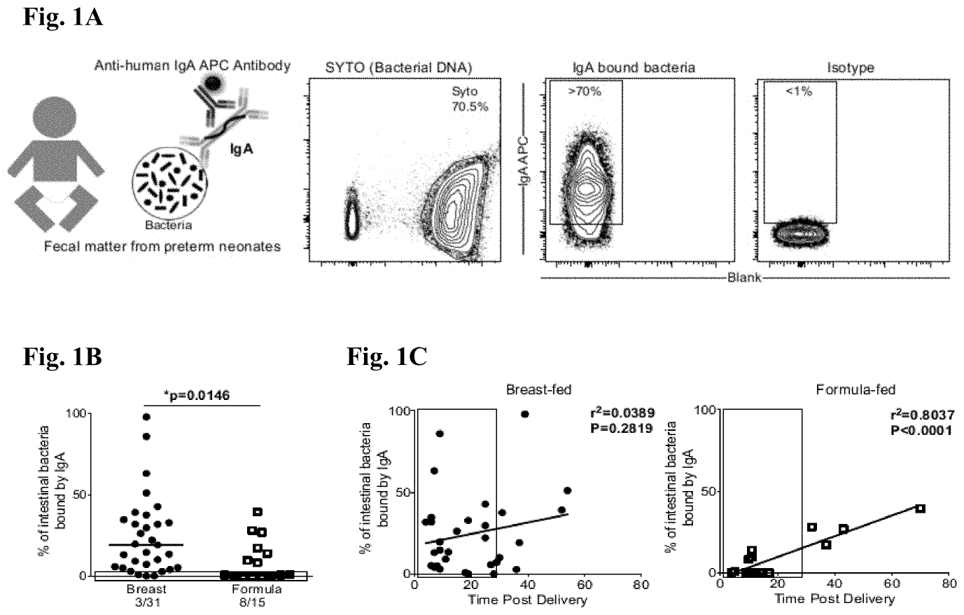

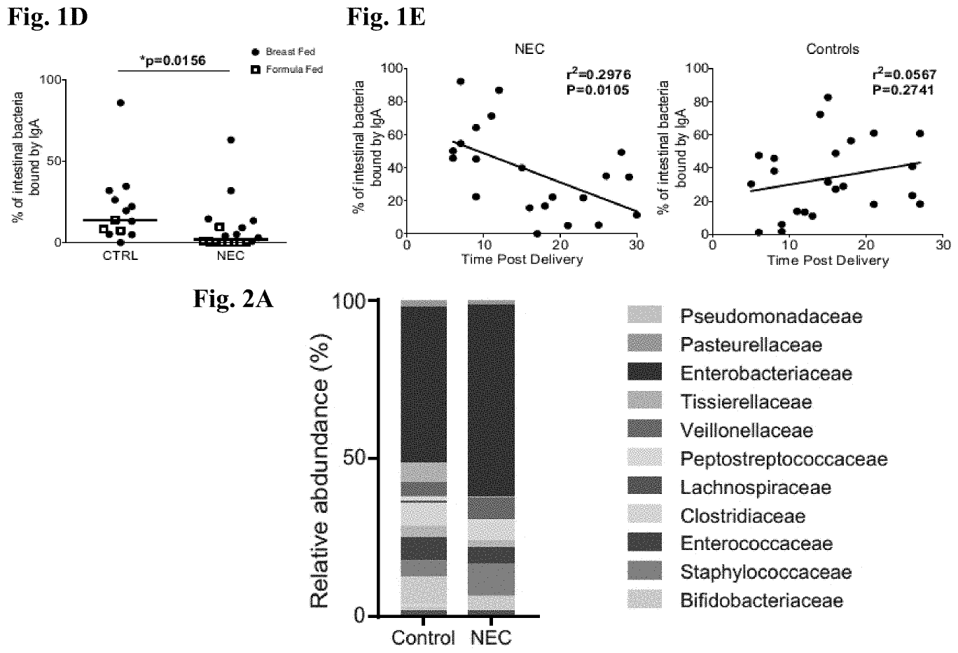

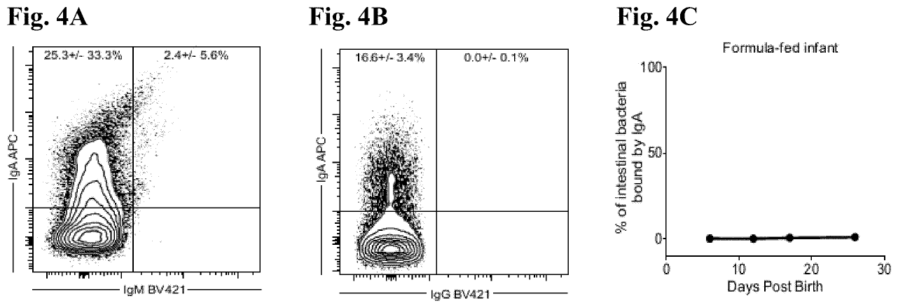

To determine whether there existed a correlation between antibody binding and the incidence of NEC, the level of immunoglobulin (Ig) binding on intestinal bacteria was analyzed from fecal samples of premature infants with NEC and age-matched healthy controls. The fecal samples were stained with anti-human IgA, IgM, and IgG antibodies and the Ig-bound population was measured by flow cytometry²³⁻²⁶. The analyses were focused on the subset of antibodies bound to bacteria in the intestine in vivo, because it allowed the study of the functional component of the Ig repertoire with regard to the microbiota. In this cohort, fecal samples from premature infants (one sample/infant; gestational age <33 weeks) obtained on day of life (DOL) 4-70 from infants diagnosed with NEC and age-matched controls (Table 1) were analyzed. Surveyed across all samples, the percentage of IgA-bound bacteria was high (FIG. 1A), but there were minimal IgM- and IgG-bound bacteria (FIGS. 4A and 4B). This result is consistent with the known role of IgA as the dominant antibody found in the intestine as the stability of IgM and IgG are limited by a lack of the covalently-bound secretory factor to protect them from proteolytic degradation^{27,28}. Therefore, the analyses on IgA became the focus.

To elucidate the relative role of maternal and infant IgA, the frequency of IgA-bound fecal bacteria from premature infants fed with formula or human breast milk was compared. These analyses identified that breast feeding was associated with a much greater frequency of IgA-bound bacteria than formula feeding and, in particular, that a majority (8/15) of formula-fed infants had <1% of their intestinal bacteria bound by IgA (FIG. 1B). Some formula-fed infants had a substantial amount of IgA-positive bacteria. Since it takes 3-4 weeks for the intestine to become populated with B cells, it was hypothesized that this was the infant's immune response against colonizing bacteria and would therefore be limited to later time points post-delivery²⁹. Indeed, a significant relationship between age post-delivery and increased IgA binding in formula-fed infants was found, but no such relationship was noticed in breast-fed infants, implying that during the first 4 weeks of life, the primary source of IgA is breast milk (FIG. 1C). Furthermore, a longitudinal study of a premature infant fed formula over the first 4 weeks of life revealed no IgA bound bacteria, strongly supporting the contention that the primary source of IgA in early life (first 30 days post-delivery) is breast milk (FIG. 4C). To focus the analysis on the effect of maternal IgA, samples collected within 30 days of birth were then analyzed. It was discovered that infants who develop NEC have, on average, a significantly lesser proportion of IgA-bound bacteria in their intestine (FIG. 1D). Thus, NEC is inversely correlated with bacterial IgA binding. However, because the group that developed NEC was much more likely to be formula fed (and thus lack intestinal IgA altogether) and these fecal samples were acquired post-diagnosis, it was important to confirm the findings in a prospective cohort where all infants were fed breast milk and not complicated by disease-associated inflammation.

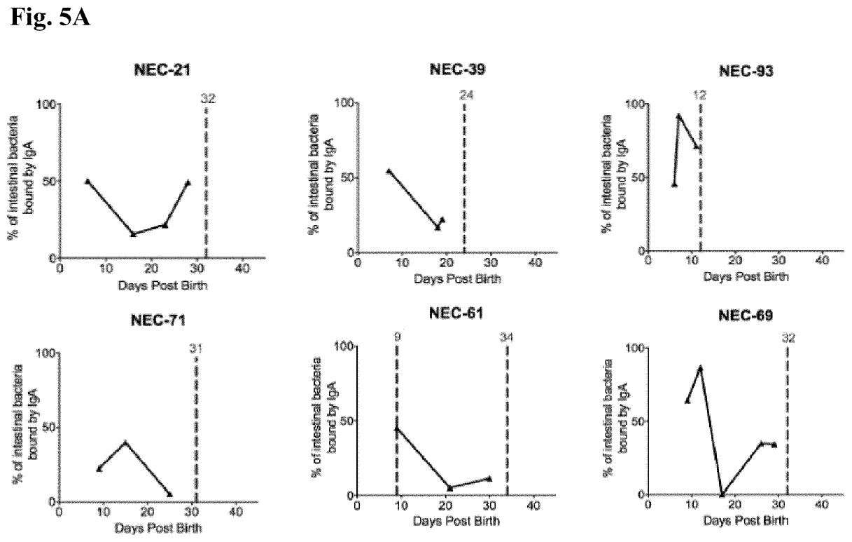

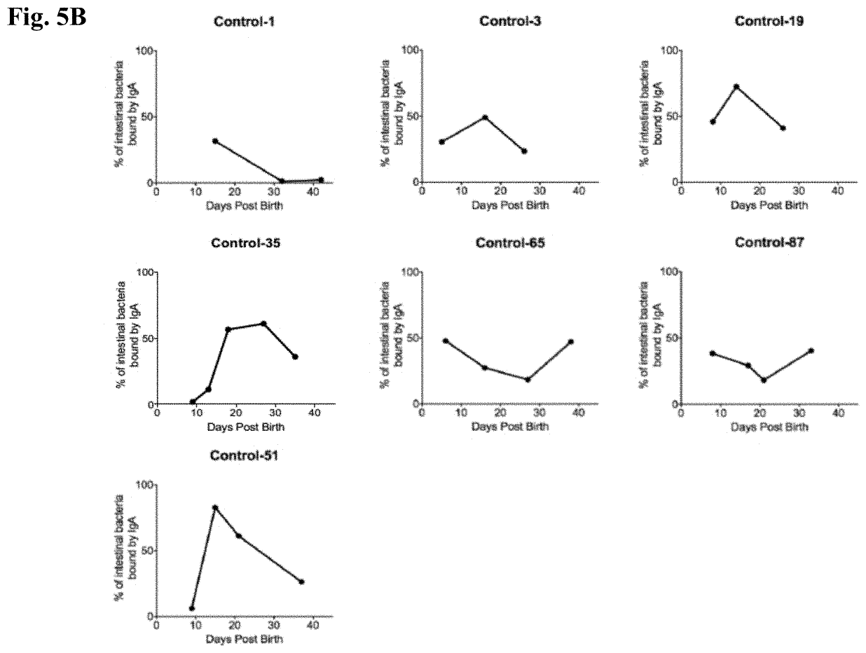

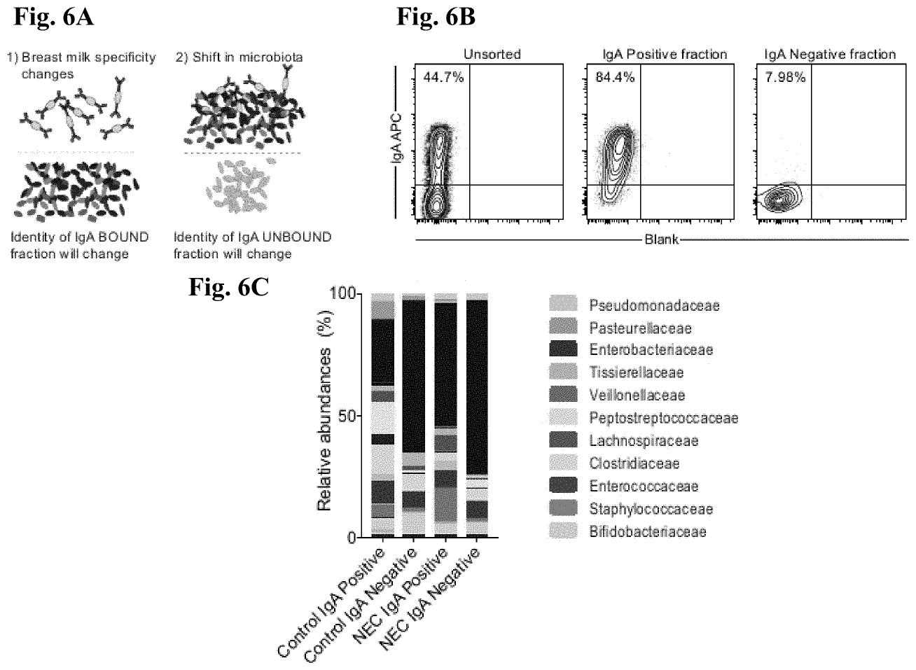

To carry out these experiments, multiple weekly prospective samples were collected from breast milk-fed preterm neonates who went on to develop NEC along with gestational age and sex matched controls (Table 2). Flow cytometric analysis of IgA binding in these samples revealed a lower percentage of IgA bound bacteria in infants in the days just before they develop NEC (days 22-30 for this cohort), which was not observed in controls (FIGS. 1E and 5). It was therefore hypothesized that the apparent loss of IgA binding on the intestinal microbiota prior to the incidence of NEC indicates either a change in IgA specificity of the breast milk or an alteration in the composition of the microbiota of the infant. These two hypotheses can be separated by identifying the IgA positive (bound) and negative (unbound) bacteria, where a change in bound bacteria implicates changes to the antibody repertoire of the breast milk, whereas changes to the unbound fraction indicates a shift in the microbiota allowing avoidance of maternal IgA (FIG. 6A). The IgA positive and negative fractions of the prospective cohort were therefore sequenced to test this hypothesis.

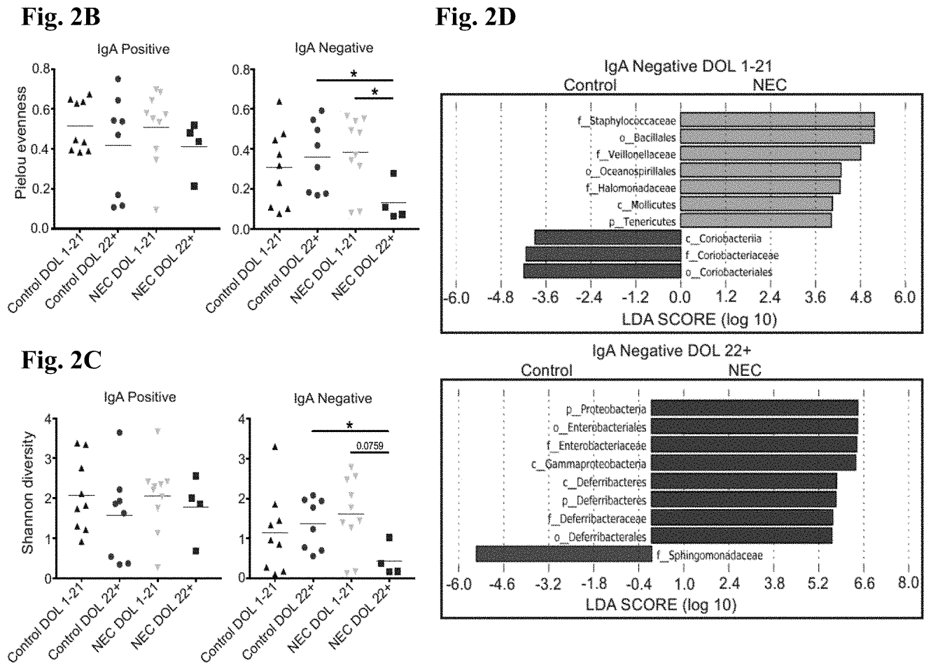

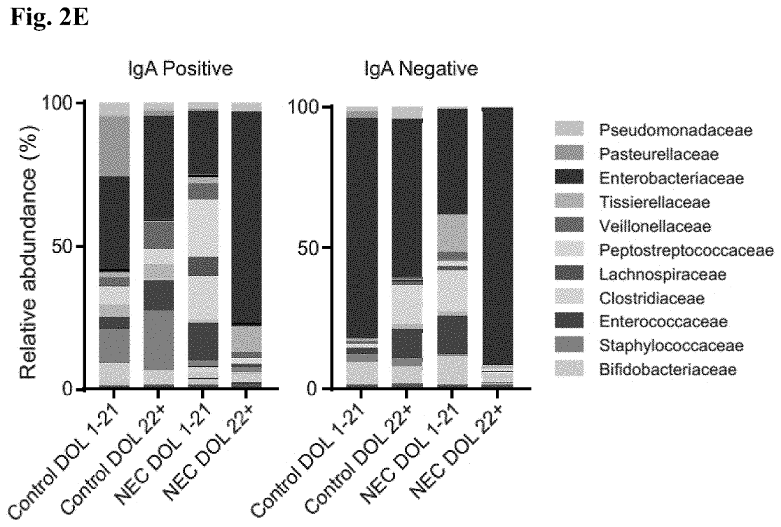

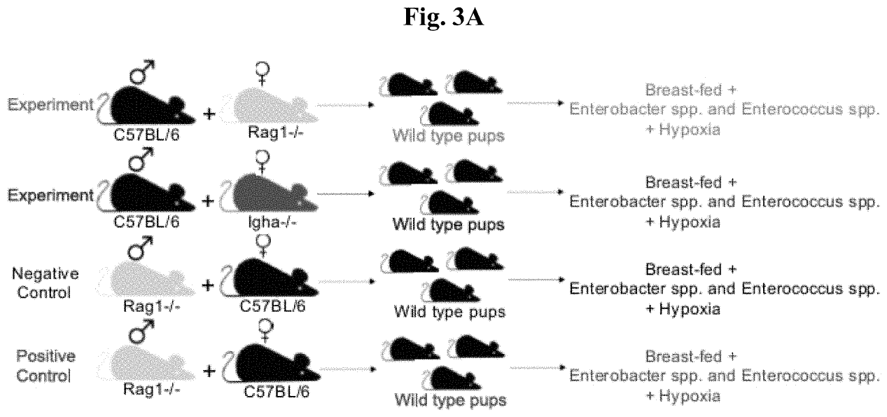

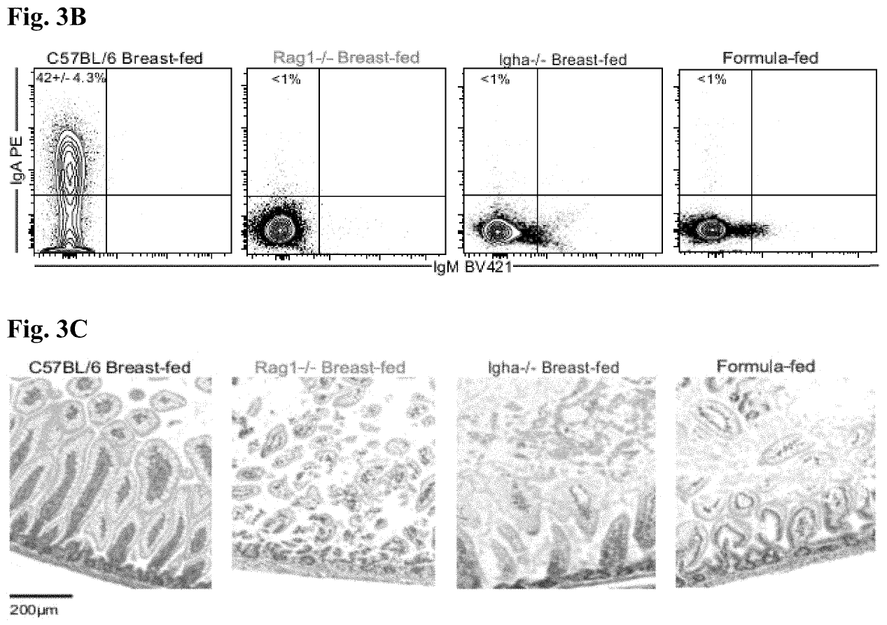

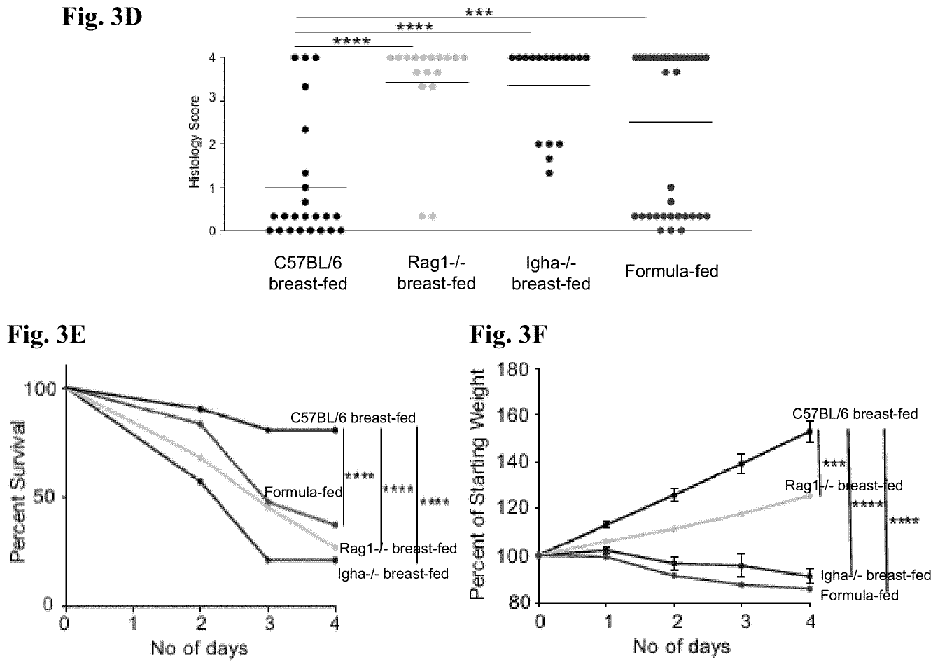

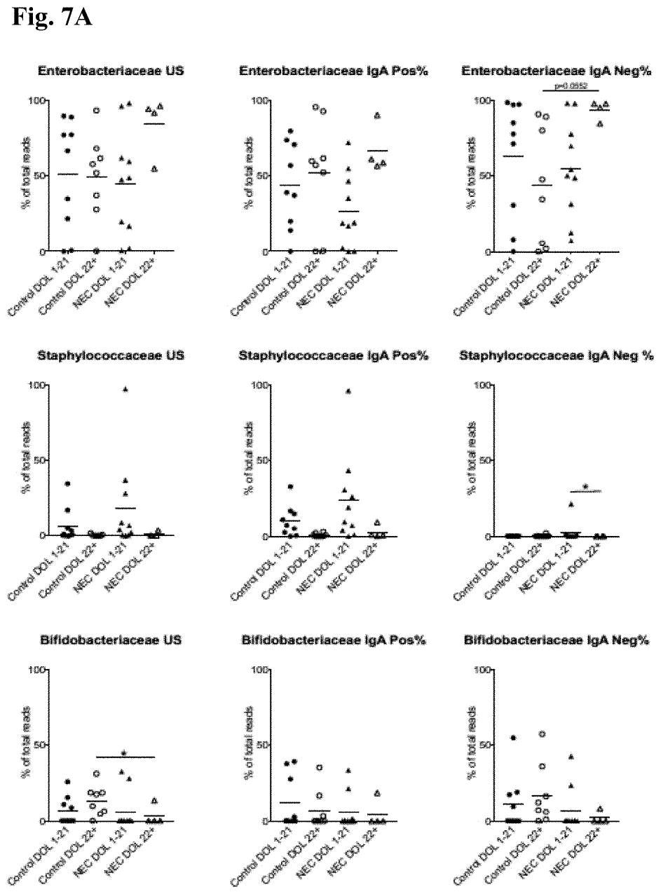

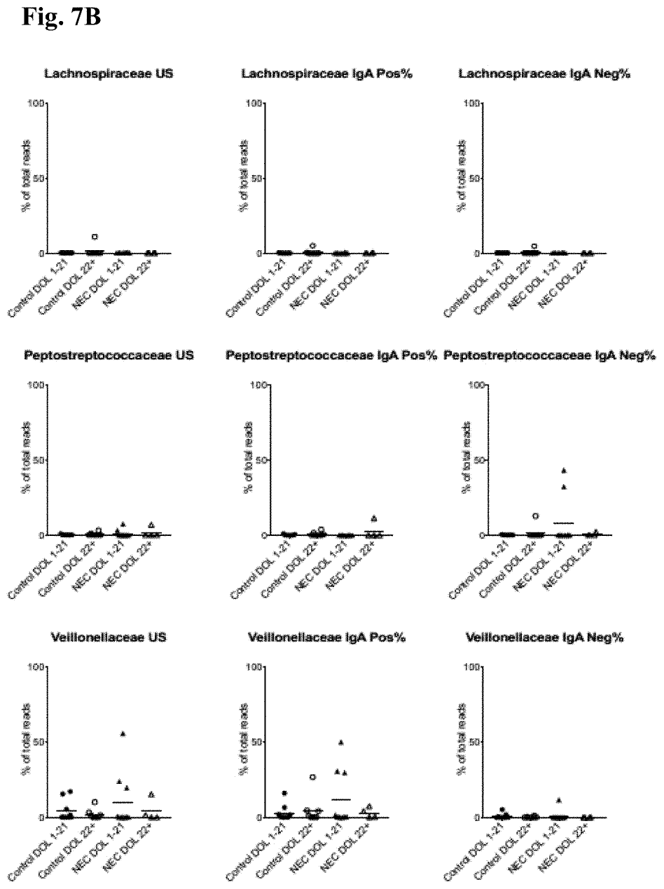

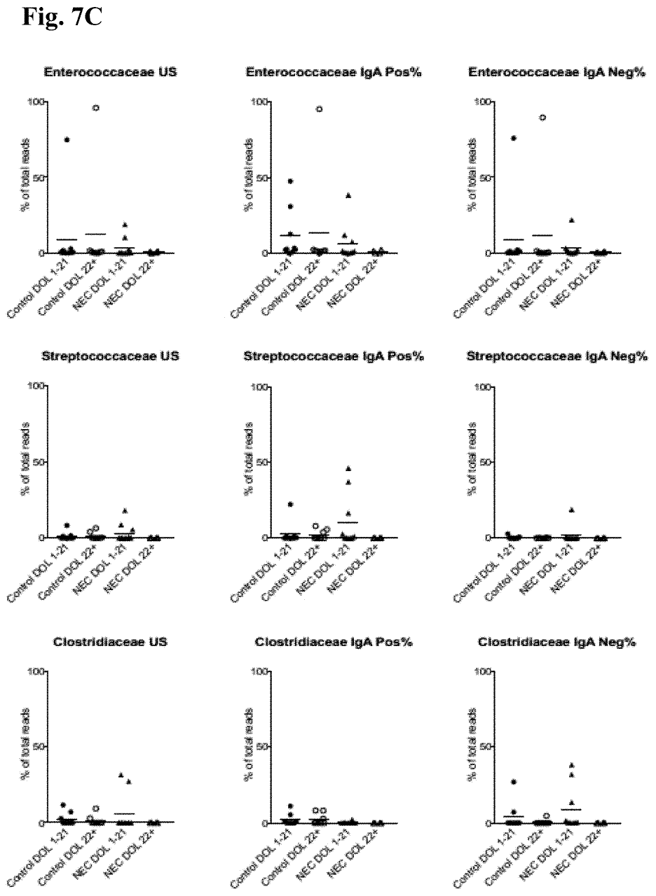

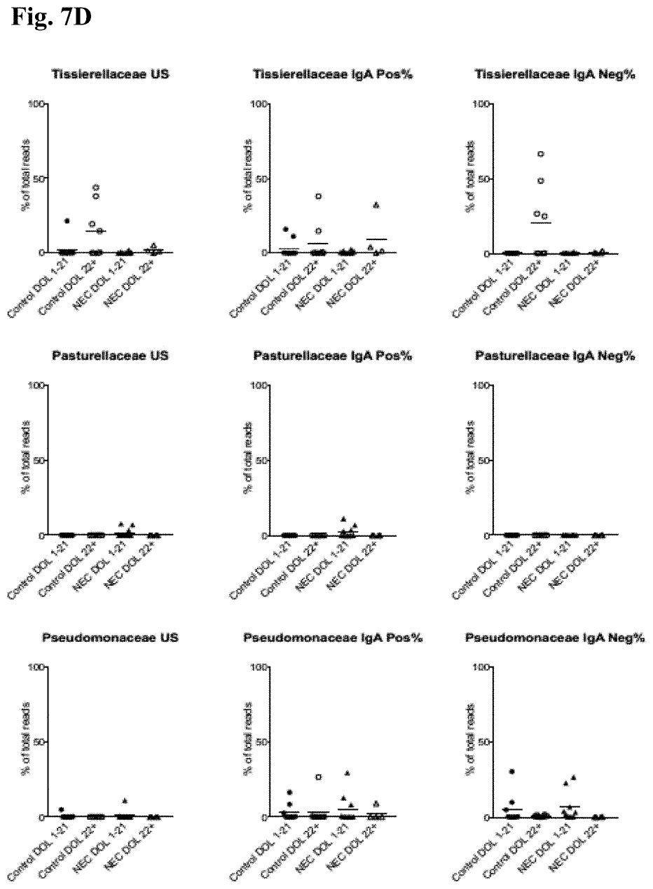

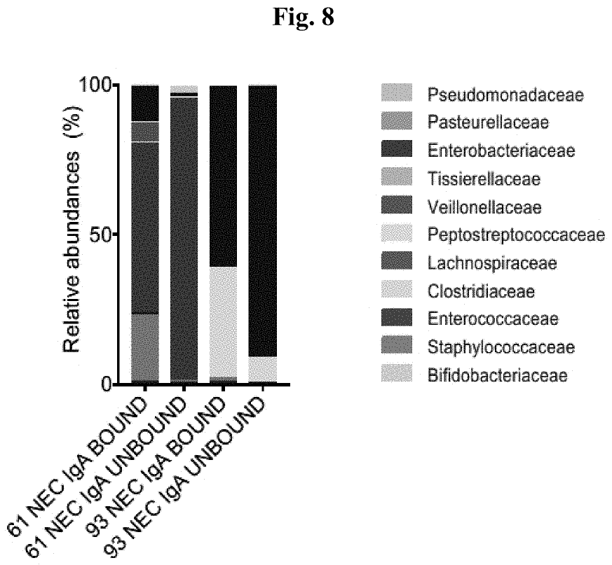

To confirm that the cohort was consistent with what has been seen in studies of the premature infant microbiome, fecal samples for microbial composition were sequenced and analyzed by analysis of 16S rRNA genes. Previously, multiple studies have identified a modest increase in Enterobacteriaceae and a modest decrease in Bifidobacteriaceae prior to the development of NEC¹⁰. Similar results in the cohort were seen, validating it as a comparable clinical group (FIG. 2A). To identify the IgA positive and negative bacteria, IgA-bound bacteria were separated using magnetic-activated cell sorting (MACS) and identified bacterial taxa by sequencing the hypervariable regions of the 16S rRNA genes (Ig-seq)^{30,31}. Perhaps due to the low frequency of IgA positive bacteria in many of the patients, >99% purity in the IgA positive/IgA negative separations was not achieved, meaning that neither the IgA positive nor the IgA negative populations could not be accurately described without “blending” of the result due to contamination. Sequencing of the input sample as well as post-sort flow cytometric analysis of each sample for IgA binding allowed us to deconvolute the contamination of the other fraction (FIG. 6B). Comparative analysis of the IgA positive and negative fractions from all patients revealed that Enterobacteriaceae was enriched in the IgA negative fraction, while Staphylococcaceae was enriched in the IgA positive fraction (FIG. 3C), indicating that Enterobacteriaceae can be unique with regard to escaping binding by the maternal IgA repertoire. Since the decrease in IgA binding was observed directly prior to the incidence of NEC, the attention was concentrated to the samples just prior to the diagnosis of NEC. Therefore, the samples were divided into two time-points based on the day of life (DOL): samples from DOL 1-21 and DOL 22+, as NEC most commonly develops during the 4^{th} week post-birth and the infants in the cohort were diagnosed 24-32 days after delivery³². After differentiation of the samples based upon DOL, a unique and significant reduction in the diversity and evenness in the IgA negative fraction of NEC DOL 22+, group were observed (FIGS. 2B and 2C). Since a change was seen only in the IgA negative fraction, this finding is consistent with the hypothesis that the decrease in the amount of IgA positive bacteria in the days leading to NEC is driven by a shift in the microbiota and not a change in maternal breast milk IgA. It was important to determine whether the loss of diversity was due to specific bacterial taxa or was unique to each infant. Linear discriminant analysis effect size (LEfSe) analysis of the bacterial taxa revealed that Enterobacteriaceae was uniquely associated with the IgA negative fraction in infants who eventually develop NEC and are DOL 22+ (FIG. 2D). Further analysis of operational taxonomic units (OTU) abundances confirmed that infants at DOL 22+ who go on to develop NEC show an increase in the relative abundance of IgA negative Enterobacteriaceae while in contrast, infants who do not progress to disease show an increased fraction of IgA positive Enterobacteriaceae as time progresses (FIGS. 2E and 7). Two infants who developed NEC during the 2^{nd} week post-delivery were not included in this analysis but, importantly, they had IgA negative fractions of very low diversity that were dominated by Enterobacteriaceae and Enterococcaceae, respectively, prior to the incidence of NEC (FIG. 8). Taken together, the data indicates that NEC is associated with a domination of the intestinal microbiota by a small group of bacteria that escape IgA binding.

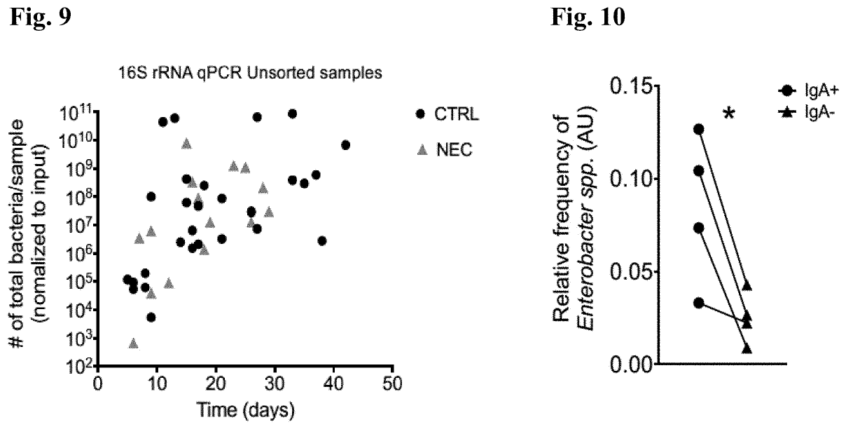

Interestingly, quantitative PCR measurement of 16S rDNA copy numbers demonstrated that the total microbial burden within fecal samples did not increase significantly prior to onset of NEC. Thus, if loss of IgA binding is associated with increased bacterial proliferation, it is not enough to significantly increase the entire population size of the intestinal microbiota (FIG. 9). The study identifies that the dominant bacteria that increase in the IgA negative fraction are Enterobacteriaceae and possibly Enterococcaceae, two families of bacteria known for their ability to thrive under inflammatory conditions^{33,34}. Thus, a model of NEC was favored where binding of specific inflammatory members of the intestinal microbiota by IgA is an important mechanism to prevent the development of NEC, partially explaining the efficacy of breast milk in protecting against the disease.

While the human studies identified potentially important associations between IgA binding of the neonatal microbiota and the development of NEC, the importance of maternal IgA was tested more directly. To do so, a commonly used murine model of experimental NEC was employed, where disease requires colonization of the intestine with Enterobacteriaceae (Enterobacter spp.; 99% of inoculum) and Enterococcaceae (Enterococcus spp.; 1% of inoculum) and feeding of 7-8 day old pups exclusively with formula. This model was well-suited to the study because breast feeding has been shown to be protective and breast-fed pups are often used as negative controls^{35,36}. To investigate the importance of IgA in NEC, a breeding program was set up, where heterozygote wild-type pups were fed by mothers that either can (C57BL/6) or cannot produce IgA (Rag1^{-/-} or Igha^{-/-}) (FIG. 3A). This scheme allows us to focus on maternally-derived intestinal IgA as pups born to wild-type C57BL/6 mothers (used as formula-fed controls) and Igha^{-/-} mothers will receive normal amounts of maternal IgG via the placenta which has been shown to be critical for control of neonatal bacteremia^{37,38}. It was confirmed that mice, like humans, produce little IgA during their first two weeks of life and that mothers are the primary source of neonatal IgA^{29,39,40} (FIG. 3B). It was also determined that Enterobacter spp. introduced into pups were enriched in the IgA positive fraction, indicating that murine dams can produce protective IgA without being vaccinated (FIG. 10). Strikingly, pups undergoing the NEC protocol that were breast-fed by mothers that lack IgA (Rag1^{-/-} or Igha^{-/-}) showed a phenotype consistent with the formula-fed pups. Specifically, they exhibited mortality and severe intestinal damage characterized by shortened and necrotic villi and mucosal sloughing that was indistinguishable from formula-fed controls (FIGS. 3C-3E). Furthermore, pups fed by Igha^{-/-} mothers that did not succumb to NEC exhibited a significant reduction in weight gain compared to pups fed by wild type mothers (FIG. 3F). Thus, it was shown using a murine model of NEC that maternal IgA provided in breast milk, is necessary for prevention of the disease.

Taken together, the work both in human premature infants and neonatal mice indicates the critical importance of maternal IgA as a preventative mechanism against the development of NEC. Of note, previous attempts to supplement the diet of at risk infants with oral Ig have largely failed to protect against the development of NEC, though the work would indicate that this is probably due to differences in the antigenic repertoire of secretory and circulatory antibodies and the unique properties of secretory IgA (sIgA)^{47,48}.

Previously it has been shown that an increased relative abundance of Enterobacteriaceae is associated with the development of NEC¹⁰. Additionally, animal studies have indicated that IgA is important in controlling Enterobacteriaceae and establishing a mature microbiota^{17,18,40}. Here the data confirms and expands the understanding of the relationship between IgA and Enterobacteriaceae and shows that in the days directly before diagnosis that many of the bacteria of this taxon escape IgA binding. Fascinatingly, the abundance of Enterobacteriaceae within the maternal intestine increases during the final trimester of pregnancy⁵⁰. One possible explanation for this increase is that the increase promotes the generation of antibody responses against this class of bacteria, of which many members are important neonatal pathogens. Thus, protection against NEC would be coincidental, and, since many infants who suffer from NEC are delivered at the beginning of the third trimester, their mothers might lack these responses.

### Example 2

The microbial specific repertoire of breast milk with regard to the surface antigens of the most important bacteria that colonize the neonatal intestine is critical to infant health, particularly in premature infants. In these infants, the incidence of NEC is associated with a significantly reduced prevalence of IgA bound bacteria, particularly those of the inflammatory Enterobacteriaceae and Enterococcaceae families. Thus, the development of this disease can be related to a “hole” in the maternal IgA repertoire with regard to the most inflammatory and invasive bacterial organisms. The impact of NEC is immense. Children diagnosed with the disease still have a mortality rate of ˜25%. Treatment of the disease often requires multiple surgeries and the cost of treating a single patient can run into the millions of dollars. Additionally, “successfully” treated patients have often had significant portions of their small intestine removed and often develop long-term consequences (such as failure to thrive) due to absorption issues. Thus, there is a critical need to prevent the disease. The only effective preventative therapy is maternal milk, which lessens the incidence of the disease-2X, though the mechanism of this protective effect is not known. Maternal IgA can also aid in protection against other clinically important neonatal infections such as Group B Streptococcus and Beta-lactam-resistant E. coli.

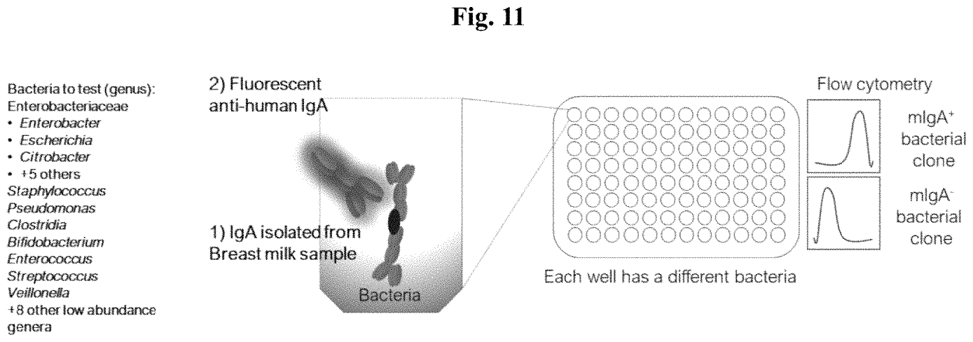

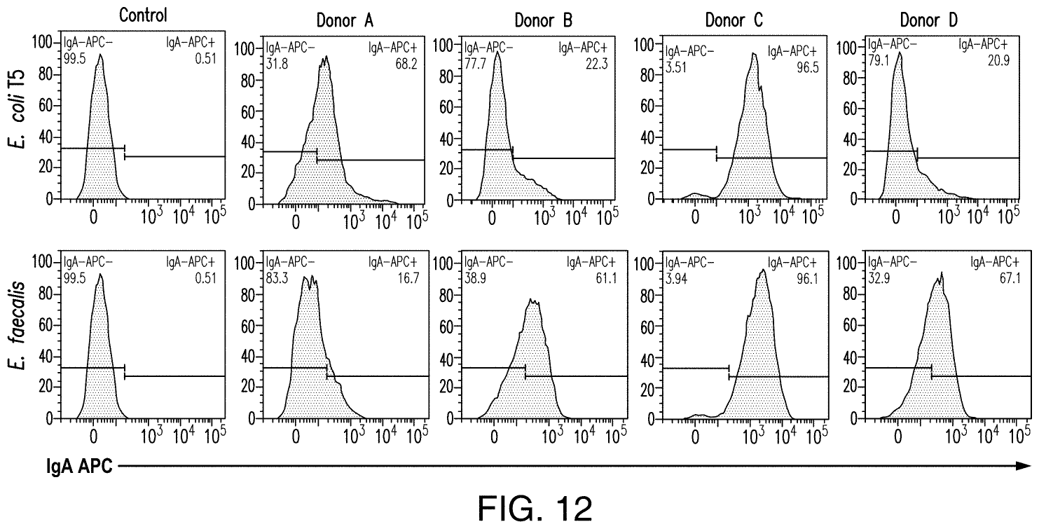

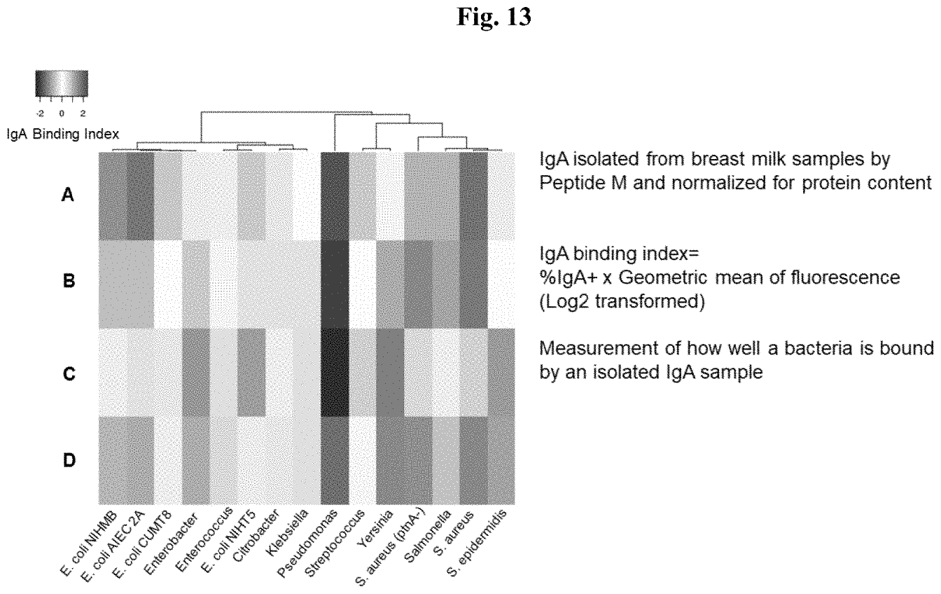

A novel method was developed to determine the anti-bacterial antibody repertoire of breast milk-derived antibodies. In short, a 96 well plate was populated with various strains of bacteria commonly found to colonize baby's intestines early after delivery (FIG. 11). The array was quarried with antibodies isolated from different mother's breast milk samples (FIG. 11). Immunoglobulin A (IgA) was tested in this Example, but this approach can be easily extended to other type of antibodies, such as IgG and IgM. The specificity of breast milk-derived antibodies for different bacterial strains was then determined by flow cytometry (FIG. 12). The flow cytometry data were quickly translated into an easy to understand heatmap of antibody binding, which provided a fingerprint of the anti-bacterial repertoire of each individual mother. An example of the heatmap is shown in FIG. 13. Each individual mother had a unique signature of IgA binding to different bacterial strains.

The method can be used to test samples from breast milk banks, which can be particularly beneficial to premature infants. The method can also be used for or further include testing viruses and intestinal parasites.

### Example 3

### NEC and Maternal Milk

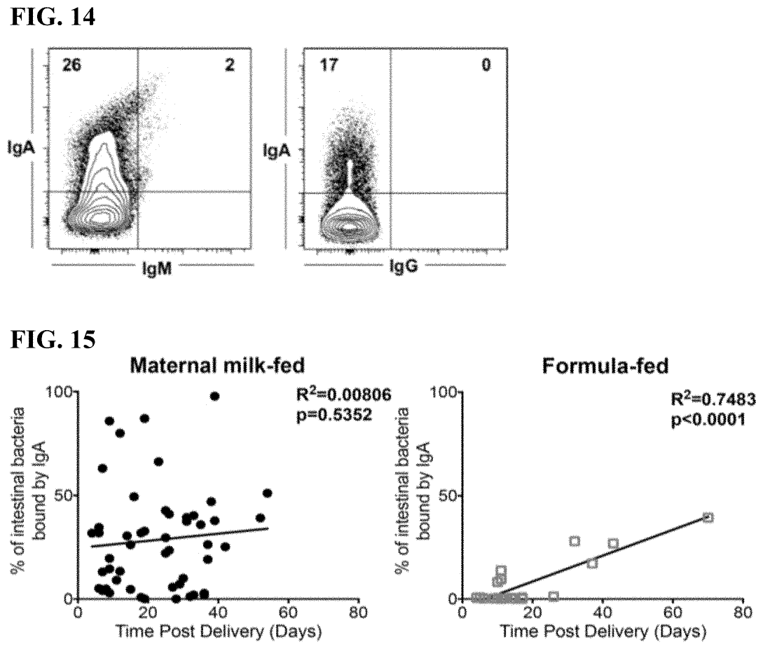

It was shown via flow cytometric analysis of IgA binding of bacteria from infant fecal samples (FIG. 14), plotted over time that preterm infants fed formula have negligible amounts of IgA on their intestinal bacteria for up to 4 weeks of age, indicating that maternal milk is the primary source of IgA post-delivery (FIG. 15). It was investigated whether IgA binding of the microbiota is important to prevent NEC.

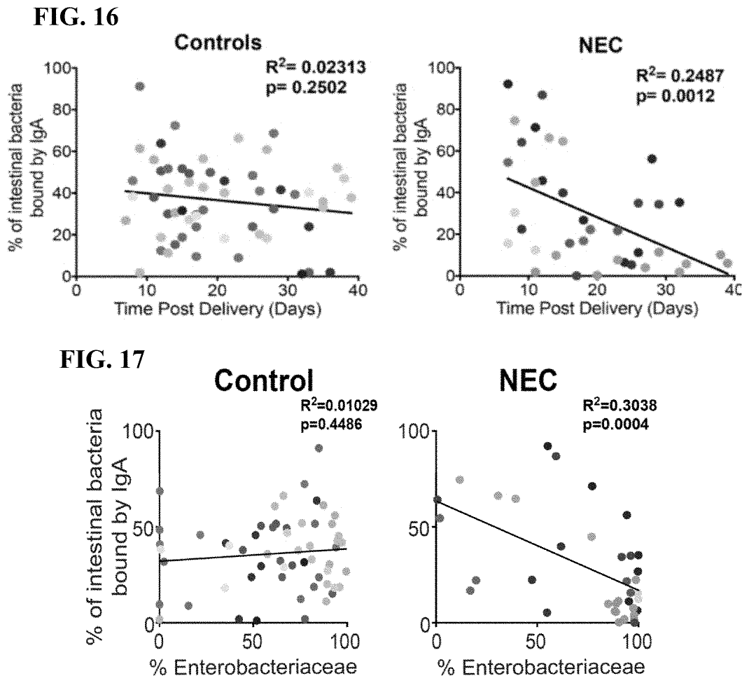

Maternal-derived IgA binding to Enterobacteriaceae is associated with protection against NEC To study the antibodies bound to the intestinal bacteria of preterm infants, fecal samples were stained with anti-human IgA, IgM, and IgG antibodies and measured the in vivo antibody-bound intestinal bacteria by flow cytometry. Analyzing antibodies actively bound to intestinal bacteria in vivo allowed us to study the functional component of the maternal Ig repertoire. Across all samples, the mean percentage of IgA-bound bacteria was high and there was much less IgM and IgG bound bacteria (FIG. 14). Prospective samples were then selected from a previously collected group of maternal milk-fed preterm infants that went on to develop NEC (along with age and sex matched controls). Flow cytometric analysis of IgA binding in these samples revealed a significant trend towards reduced IgA binding of intestinal bacteria in the time (post-gestation; day 15-30) most commonly associated with the development of NEC in affected infants that was not observed in controls (FIG. 16). Interestingly, the percent IgA bound bacteria was also inversely proportional to the abundance of

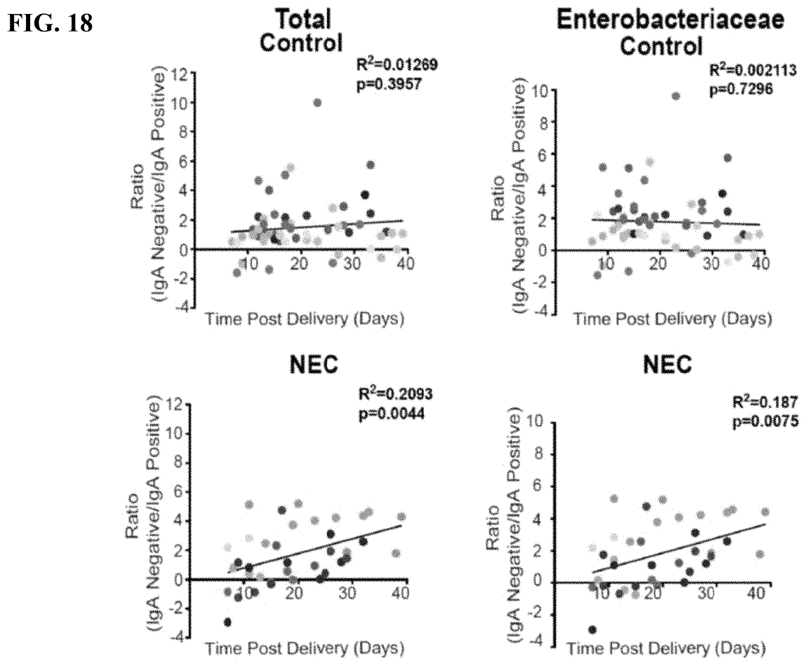

Enterobacteriaceae family bacteria, but only in infants that will develop NEC, possibly indicating that this taxa contributes to the observed drop in IgA⁺ bacteria (FIG. 17). Next, it was determined which bacterial taxa are bound by maternal antibodies in the prospective cohort to identify the taxa “escaping” IgA binding. To identify the IgA⁺ and IgA⁻ bacteria, the bacteria were separated using magnet-activated cell sorting (MACS) and bacterial taxa was measured by NextGen Sequencing (IgSeq). Any post-sort contamination was corrected for using a novel in silica deconvolution technique that aligns the IgA⁺ and IgA⁻ 16S rRNA gene read number post sequencing with the percent IgA⁺ bacteria measured in the total sample by flow cytometry (FIG. 16). This innovation also the comparison between the IgA⁺ and IgA⁻ fractions of bacteria quantitatively. The ratio of reads between IgA⁺ and IgA⁻ samples were compared, and an increase in IgA⁻ 16S rRNA reads was observed uniquely in NEC patients, similar to the flow cytometrics (FIGS. 16 AND 18). Critically, it was also observed that the relative increase in IgA⁻ bacteria is driven by loss of IgA binding on a single taxon, Enterobacteriaceae, and no other major taxa of the preterm infant (FIG. 18). Thus, a decrease in the prevalence of IgA-bound Enterobacteriaceae was detected in the time period directly prior to the development of NEC unique to those infants that will be affected.

### 2019 Heterogeneity of Maternal IgA

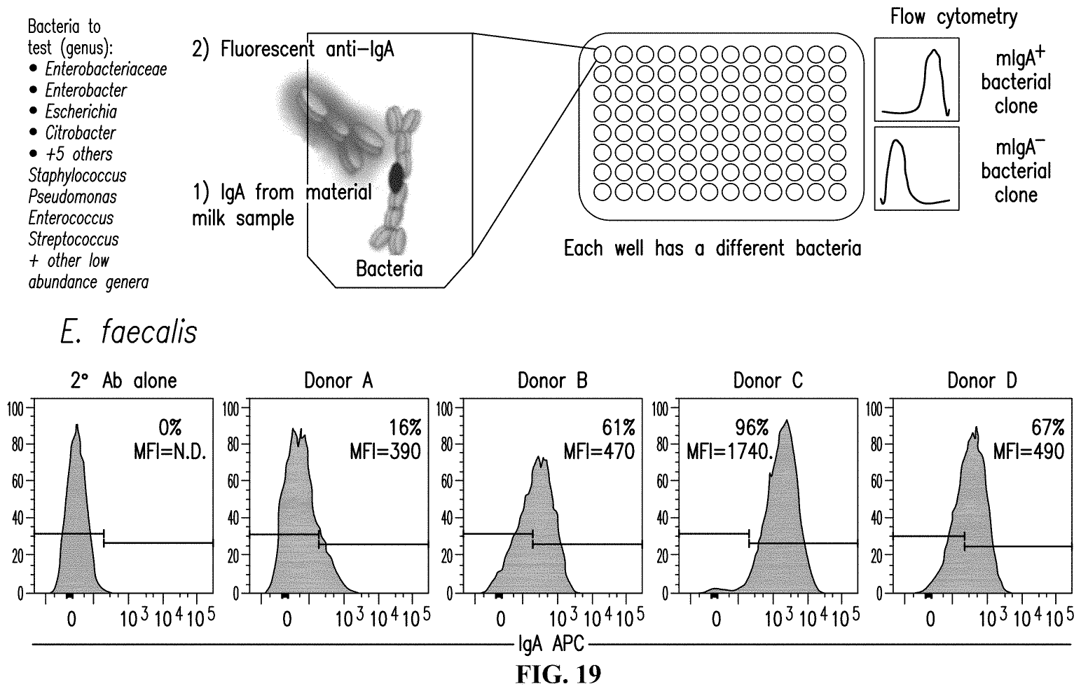

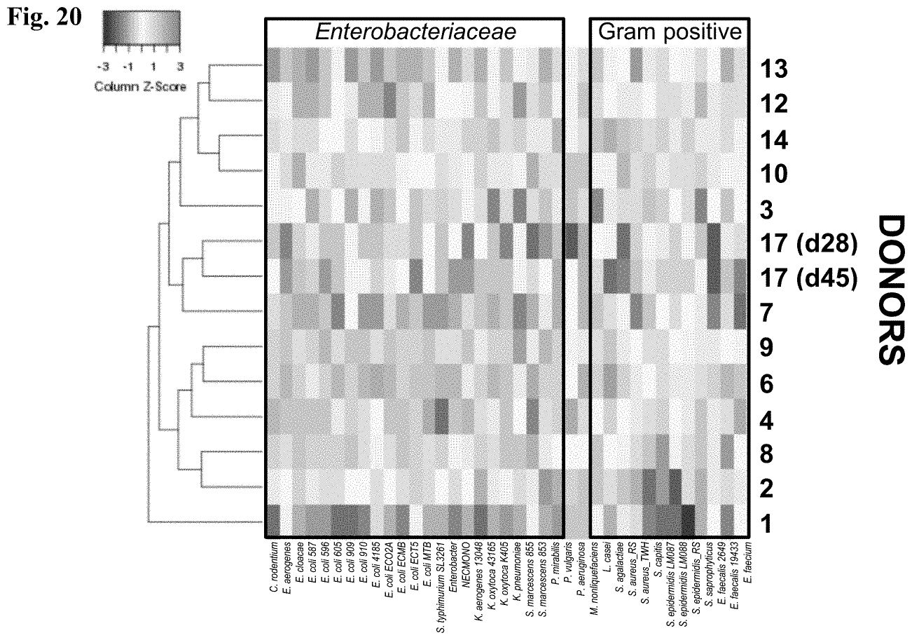

Identical B cell clones have been found in the mammary glands and GI tracts of mice, implying that microbiota-specific B cells traffic from the intestine to the mammary gland during pregnancy. Since the specificity of intestinal B cells is shaped by the microbiota and history of GI infection, the IgA repertoire of maternal milk can also depend on the intestinal microbiota of the mother and thus can vary significantly from individual to individual. To test this, the novel flow cytometric bacterial array of FIG. 19 was developed and used to determine the specificity of IgA isolated by Peptide M columns from donor maternal milk samples. Using this array, it was shown that there is significant heterogeneity between the IgA specificities of maternal milk from different donors, but that they can be separated into those with broad specificity against Enterobacteriaceae, those that skew towards specificity against Gram positive bacteria such as Staphylococcaceae and those that have weak responses against all bacteria (FIG. 20).

### Maternal IgA is Necessary to Protect Pups in the Murine Model of NEC

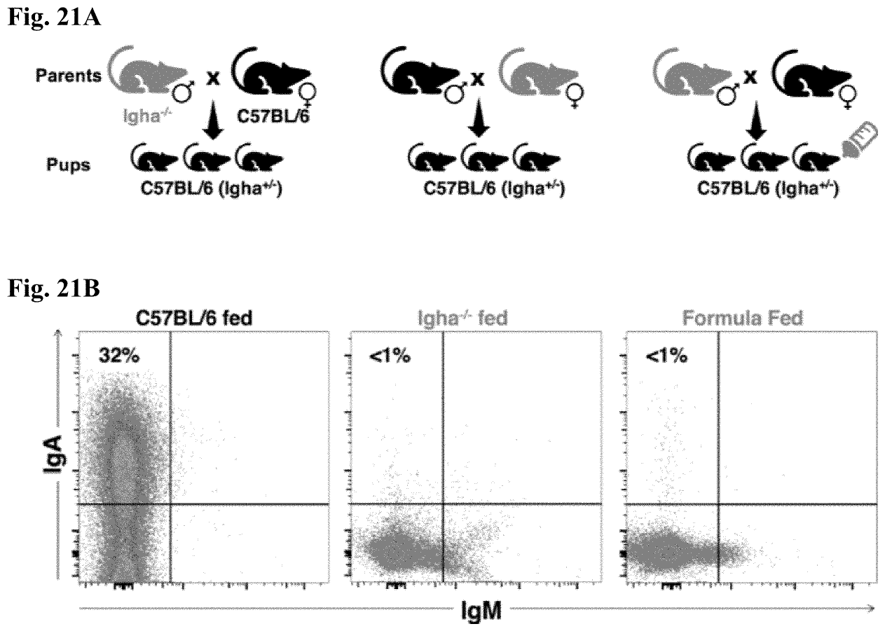

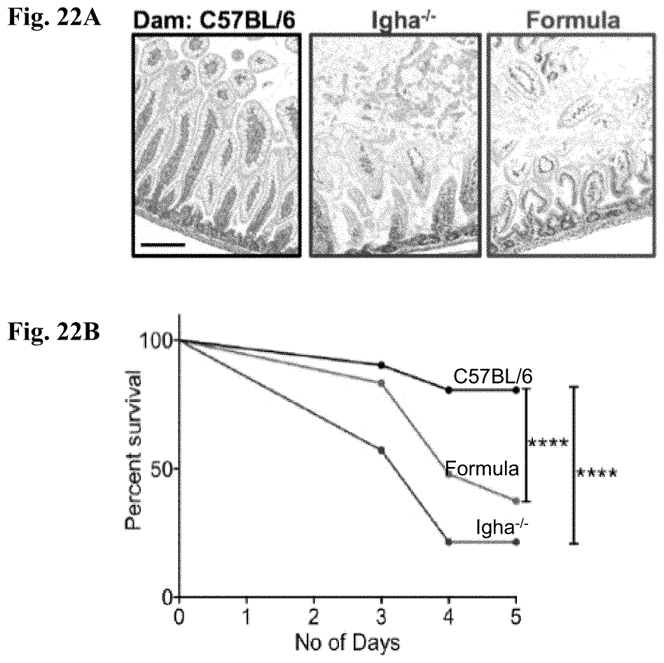

The experimental mouse model of NEC recapitulates human disease in many aspects and is useful as mice are born with intestines that developmentally resemble preterm infants. To test whether mIgA is necessary for protection, a breeding system was set up wherein IgA would specifically be removed from the feeds of isogenic mouse pups (FIG. 21A). Some pups in every experiment were also separated from their mothers and hand-fed formula as positive controls. It was confirmed that mice produce little IgA during their first two weeks of life and that mIgA is the primary source of IgA (FIG. 21B). Strikingly, pups undergoing the NEC protocol that are breast-fed by mothers that lack IgA (Igha^{-/-}) exhibited mortality and severe intestinal damage characterized by shortened and necrotic villi and mucosal sloughing indistinguishable from formula-fed controls (FIGS. 22A and 22B). Thus, it was shown using a murine model of NEC that mIgA provided in maternal milk is necessary for the prevention of disease. This model provides the ability to test the sufficiency of specific antibodies in an animal model.

### REFERENCES

1. Neu, J. & Walker, W. A. Necrotizing enterocolitis. N Engl J Med 364, 255-264, doi:10.1056/NEJMra1005408 (2011).

2. Hintz, S. R. et al. Neurodevelopmental and growth outcomes of extremely low birth weight infants after necrotizing enterocolitis. Pediatrics 115, 696-703 (2005).

3. Neu, J. & Pammi, M. in Seminars in perinatology. 29-35 (Elsevier).

4. Hackam, D. & Caplan, M. in Seminars in pediatric surgery. 11-18 (Elsevier).

5. Martin, C. R. & Walker, W. A. in Seminars in Fetal and Neonatal Medicine. 369-377 (Elsevier).

6. Good, M. et al. Breast milk protects against the development of necrotizing enterocolitis through inhibition of Toll-like receptor 4 in the intestinal epithelium via activation of the epidermal growth factor receptor. Mucosal immunology 8, 1166 (2015).

7. Meinzen-Derr, J. et al. Role of human milk in extremely low birth weight infants' risk of necrotizing enterocolitis or death. Journal of Perinatology 29, 57 (2009).

8. Lucas, A. & Cole, T. Breast milk and neonatal necrotising enterocolitis. The Lancet 336, 1519-1523 (1990).

9. Cortez, J. et al. Maternal milk feedings reduce sepsis, necrotizing enterocolitis and improve outcomes of premature infants. Journal of Perinatology 38, 71 (2018).

10. Pammi, M. et al. Intestinal dysbiosis in preterm infants preceding necrotizing enterocolitis: a systematic review and meta-analysis. Microbiome 5, 31 (2017).

11. Brower-Sinning, R. et al. Mucosa-associated bacterial diversity in necrotizing enterocolitis. PloS one 9, e105046 (2014).

12. Le Doare, K., Holder, B., Bassett, A. & Pannaraj, P. S. Mother's milk: A purposeful contribution to the development of the infant microbiota and immunity. Frontiers in immunology 9, 361 (2018).

13. Good, M. et al. The human milk oligosaccharide 2′-fucosyllactose attenuates the severity of experimental necrotising enterocolitis by enhancing mesenteric perfusion in the neonatal intestine. British Journal of Nutrition 116, 1175-1187 (2016).

14. Jantscher-Krenn, E. et al. The human milk oligosaccharide disialyllacto-N-tetraose prevents necrotising enterocolitis in neonatal rats. Gut, gutjnl-2011-301404 (2011).

15. Wisgrill, L. et al. Human lactoferrin attenuates the proinflammatory response of neonatal monocyte-derived macrophages. Clinical & Experimental Immunology (2018).

16. Brandtzaeg, P. The mucosal immune system and its integration with the mammary glands. The Journal of pediatrics 156, S8-S15 (2010).

17. Mirpuri, J. et al. Proteobacteria-specific IgA regulates maturation of the intestinal microbiota. Gut Microbes 5, 28-39, doi:10.4161/gmic.26489 (2014).

18. Planer, J. D. et al. Development of the gut microbiota and mucosal IgA responses in twins and gnotobiotic mice. Nature 534, 263 (2016).

19. Roux, M., Mcwilliams, M., Phillips-Quagliata, J. M., Weisz-Carrington, P. & Lamm, M. Origin of IgA-secreting plasma cells in the mammary gland. Journal of experimental medicine 146, 1311-1322 (1977).

20. Wilson, E. & Butcher, E. C. CCL28 controls immunoglobulin (Ig) A plasma cell accumulation in the lactating mammary gland and IgA antibody transfer to the neonate. Journal of Experimental Medicine 200, 805-809 (2004).

21. Lindner, C. et al. Diversification of memory B cells drives the continuous adaptation of secretory antibodies to gut microbiota. Nature immunology 16, 880 (2015).

22. Arboleya, S. et al. Impact of Prematurity and Perinatal Antibiotics on the Developing Intestinal Microbiota: A Functional Inference Study. Int J Mol Sci 17, doi:10.3390/ijms17050649 (2016).

23. Kubinak, J. L. et al. MyD88 signaling in T cells directs IgA-mediated control of the microbiota to promote health. Cell host & microbe 17, 153-163 (2015).

24. Cullender, T. C. et al. Innate and adaptive immunity interact to quench microbiome flagellar motility in the gut. Cell host & microbe 14, 571-581 (2013).

25. Palm, N. W. et al. Immunoglobulin A coating identifies colitogenic bacteria in inflammatory bowel disease. Cell 158, 1000-1010, doi:10.1016/j.cell.2014.08.006 (2014).

26. Slack, E. et al. Innate and adaptive immunity cooperate flexibly to maintain host-microbiota mutualism. Science 325, 617-620, doi:325/5940/617 [pii]10.1126/science.1172747 (2009).

27. Brown, W. R., Newcomb, R. W. & Ishizaka, K. Proteolytic degradation of exocrine and serum immunoglobulins. The Journal of clinical investigation 49, 1374-1380 (1970).

28. Lindh, E. Increased resistance of immunoglobulin A dimers to proteolytic degradation after binding of secretory component. The journal of immunology 114, 284-286 (1975).

29. Rognum, T. O., Thrane, P. S., Stoltenberg, L., Vege, Å. & Brandtzaeg, P. Development of intestinal mucosal immunity in fetal life and the first postnatal months. Pediatric research 32, 145 (1992).

30. Palm, N. W. et al. Immunoglobulin A coating identifies colitogenic bacteria in inflammatory bowel disease. Cell 158, 1000-1010 (2014).

31. Bunker, J. J. et al. Innate and adaptive humoral responses coat distinct commensal bacteria with immunoglobulin A. Immunity 43, 541-553 (2015).

32. Yee, W. H. et al. Incidence and timing of presentation of necrotizing enterocolitis in preterm infants. Pediatrics 129, e298-e304 (2012).

33. Brandl, K. et al. Vancomycin-resistant enterococci exploit antibiotic-induced innate immune deficits. Nature 455, 804 (2008).

34. Winter, S. E. et al. Host-derived nitrate boosts growth of E. coli in the inflamed gut. science 339, 708-711 (2013).

35. Barlow, B. et al. An experimental study of acute neonatal enterocolitis—the importance of breast milk. Journal of pediatric surgery 9, 587-595 (1974).

36. Jilling, T. et al. The roles of bacteria and TLR4 in rat and murine models of necrotizing enterocolitis. The Journal of Immunology 177, 3273-3282 (2006).

37. Zeng, M. Y. et al. Gut microbiota-induced immunoglobulin G controls systemic infection by symbiotic bacteria and pathogens. Immunity 44, 647-658 (2016).

38. Koch, M. A. et al. Maternal IgG and IgA antibodies dampen mucosal T helper cell responses in early life. Cell 165, 827-841 (2016).

39. Harris, N. L. et al. Mechanisms of neonatal mucosal antibody protection. J Immunol 177, 6256-6262 (2006).

40. Rogier, E. W. et al. Secretory antibodies in breast milk promote long-term intestinal homeostasis by regulating the gut microbiota and host gene expression. Proc Natl Acad Sci USA 111, 3074-3079, doi:10.1073/pnas.1315792111 (2014).

41. Moor, K. et al. High-avidity IgA protects the intestine by enchaining growing bacteria. Nature 544, 498 (2017).

42. Peterson, D. A., McNulty, N. P., Guruge, J. L. & Gordon, J. I. IgA response to symbiotic bacteria as a mediator of gut homeostasis. Cell Host Microbe 2, 328-339, doi:10.1016/j.chom.2007.09.013 (2007).

43. Bunker, J. J. et al. Natural polyreactive IgA antibodies coat the intestinal microbiota. Science 358, eaan6619 (2017).

44. Quan, C. P., Berneman, A., Pires, R., Avrameas, S. & Bouvet, J. P. Natural polyreactive secretory immunoglobulin A autoantibodies as a possible barrier to infection in humans. Infect Immun 65, 3997-4004 (1997).

45. Suzuki, K., Maruya, M., Kawamoto, S. & Fagarasan, S. Roles of B-1 and B-2 cells in innate and acquired IgA-mediated immunity. Immunological reviews 237, 180-190 (2010).

46. Benckert, J. et al. The majority of intestinal IgA+ and IgG+ plasmablasts in the human gut are antigen-specific. J Clin Invest 121, 1946-1955, doi:10.1172/JCI44447 (2011).

47. Eibl, M. M., Wolf, H. M., FUrnkranz, H. & Rosenkranz, A. Prevention of necrotizing enterocolitis in low-birth-weight infants by IgA-IgG feeding. New England Journal of Medicine 319, 1-7 (1988).

48. Foster, J. P., Seth, R. & Cole, M. J. Oral immunoglobulin for preventing necrotizing enterocolitis in preterm and low birth weight neonates. Cochrane Database Syst Rev 4, CD001816, doi:10.1002/14651858.CD001816.pub3 (2016).

49. Morowitz, M. J. et al. Strain-resolved community genomic analysis of gut microbial colonization in a premature infant. Proceedings of the National Academy of Sciences 108, 1128-1133 (2011).

50. Koren, O. et al. Host remodeling of the gut microbiome and metabolic changes during pregnancy. Cell 150, 470-480 (2012).

51. Caporaso, J. G. et al. Ultra-high-throughput microbial community analysis on the Illumina HiSeq and Mi Seq platforms. The ISMS journal 6, 1621 (2012).

52. Caporaso, J. G. et al. Global patterns of 16S rRNA diversity at a depth of millions of sequences per sample. Proceedings of the National Academy of Sciences 108, 4516-4522 (2011).

53. Edgar, R. C. UPARSE: highly accurate OTU sequences from microbial amplicon reads. Nat Methods 10, 996-998, doi:10.1038/nmeth.2604 (2013).

54. Caporaso, J. G. et al. QIIME allows analysis of high-throughput community sequencing data. Nat Methods 7, 335-336, doi:10.1038/nmeth.f.303 (2010).

55. Segata, N. et al. Metagenomic biomarker discovery and explanation. Genome Biol 12, R60, doi:10.1186/gb-2011-12-6-r60 (2011).

56. Patel, C. B., Shanker, R., Gupta, V. K. & Upadhyay, R. S. Q-PCR Based Culture-Independent Enumeration and Detection of Enterobacter: An Emerging Environmental Human Pathogen in Riverine Systems and Potable Water. Front Microbiol 7, 172, doi:10.3389/fmicb.2016.00172 (2016).

57. Good, M. et al. Lactobacillus rhamnosus HN001 decreases the severity of necrotizing enterocolitis in neonatal mice and preterm piglets: evidence in mice for a role of TLR9. Am J Physiol Gastrointest Liver Physiol 306, G1021-1032, doi:10.1152/ajpgi.00452.2013 (2014).

58. Radulescu, A. et al. Heparin-binding epidermal growth factor-like growth factor overexpression in transgenic mice increases resistance to necrotizing enterocolitis. Journal of pediatric surgery 45, 1933-1939 (2010).

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A method of detecting an antibody in a sample, the method comprising:
(a) contacting a bacterial array with the sample, wherein the bacteria array comprises bacteria that are associated with necrotizing enterocolitis (NEC); and
(b) detecting binding of an antibody of the sample to a bacterium of the bacterial array.

2. The method of claim 1, further comprising isolating an antibody repertoire comprising the antibody.

3. The method of claim 1, wherein following detection of the antibody in the sample, the sample is selected to be administered to an infant.

4. A method of treating or preventing NEC in an infant in need thereof, the method comprising administering a composition comprising an antibody that binds to a bacterium associated with NEC.

5. The method of claim 4, wherein the composition is milk or infant formula.

6. The method of claim 5, wherein the milk is a breast milk.

7. The method of claim 6, wherein the breast milk is human breast milk.

8. The method of claim 4, wherein the antibody is an immunoglobulin.

9. The method of claim 4, wherein the antibody is an IgA class antibody.

10. The method of claim 4, wherein the antibody is a secretory IgA (sIgA) antibody.

11. The method of claim 4, wherein the antibody binds to bacteria of one or more family selected from the group consisting of Enterobacteriaceae, Pasteurellaceae, Pseudomonadaceae, Tissierellaceae, Veillonellaceae, Peptostreptococcaceae, Lachnospiraceae, Clostridiaceae, Enterococcaceae, Staphylococcaceae, Streptococcoceae and Bifidobacteriaceae.

12. The method of claim 4, wherein the antibody binds to bacteria of one or more genus selected from the group consisting of Enterobacter, Escherichia, Citrobacter, Salmonella, Klebsiella, Corynebacterium, Lactobacillus, Proteus, Haemophilus, Staphylococcus, Pseudomonas, Clostridium, Bifidobacterium, Enterococcus, Streptococcus and Veillonella.

13. The method of claim 4, wherein the antibody binds to one or more bacterium selected from the group consisting of E. coli NIHMB, E. coli AIEC 2A, E. coli CUMT8, Enterobacter, Enterococcus, E. coli NIHT5, Citrobacter, Klebsiella, Pseudomonas, Streptococcus, Yersinis, S. aureus (ptnA), Salmonella, S. aureus, Enterobacter (NECteria monoculture), Escherichia (ECORL), Escherichia (ECMB), Escherichia (ECT5), Escherichia (MT8), Escherichia coli 909, Escherichia coli 910, Escherichia coli 4185, Citrobacter rodentium 51459, Salmonella typhimurium 3261, Klebsiella pneumoniae, Enterobacter cloacae, Klebsiella oxytoca, Klebsiella aerogenes, Corynebacterium spp., Lactobacillus casei, Lactobacillus paracasei, Proteus mirabilis, Streptococcus agalactiae, Streptococcus salivarius, Streptococcus pneumoniae 19F, Veillonella parvula, Veillonella atypica, Enterococcus faecalis 19433, Enterococcus faecalis 286, Enterococcus faecium, Bifidobacterium breve, Bifidobacterium bifidum, Clostridium butyricum, Clostridium sporogenes, Clostridium perfringens, Pseudomonas aerginosa 01, Staphylococcus aureus, Staph aureus Protein A-, MSSA NARSA227, MSSA NARSA235, Staphylococcus capitis, Staph epidermidis B1468, Staph epi. NARSA101, Staph epi. NIHLM087, Staph saprophyticus, Haemophilus parainfluenza, and S. epidermidis.

14. The method of claim 4, wherein the antibody binds to one or more bacterium selected from the group consisting of C. rodentium, E. aerogenes, E. cloacae, E. coli 587, E. coli 596, E. coli 605, E. coli 909, E. coli 910, E. coli 4185, E. coli ECO2A, E. coli ECMB, E. coli ECT5, E. coli MTB, S. typhimurium SL3261, Enterobacter NECMONO, K. aerogenes 13048, K. oxytoca 43165, K. oxytoca K405, K. pneumoniae, S. marcescens 855, S. marcescens 853, P. mirabilis, P. vulgaris, P. aeruginosa, M. nonliquefaciens, L. casei, S. agalactiae, S. aureus_RS, S. aureus_TWH, S. capitis, S. epidermidis LM087 , S. epidermidis LM088, S. epidermidis_RS, S. saprophyticus, E. faecalis 2649, E. faecalis 19433, and E. faecium.

15. The method of claim 4, wherein the infant is a premature infant.

16. A method for treating or preventing NEC in an infant in need thereof, the method comprising:
administering to the infant an effective amount of milk or an infant formula comprising an effective amount of an antibody that binds to one or more bacterium from the family selected from the group consisting of Enterobacteriaceae, Streptococcoceae, Veillonellaceae, Enterococcaceae, Bifidobacteriaceae, Clostridiaceae, Pseudomonadaceae, Staphylococcaceae, Pasteurellaceae, and a combination thereof

17. The method of claim 16, wherein the milk is breast milk.

18. The method of claim 17, wherein the breast milk is human breast milk.

19. The method of claim 16, wherein the antibody is a secretory IgA (sIgA) antibody.

20. The method of claim 16, wherein the infant is a premature infant.


SEQUENCE LISTING (4 sequences)

SEQ ID NO: 1 - DNA, 21 bp, Artificial Sequence
  Synthetic construct
actcctacgggaggcagcagt

SEQ ID NO: 2 - DNA, 18 bp, Artificial Sequence
  Synthetic construct
attaccgcggctgctggc

SEQ ID NO: 3 - DNA, 20 bp, Artificial Sequence
  Synthetic construct
agtggaacggtctggaaagg

SEQ ID NO: 4 - DNA, 23 bp, Artificial Sequence
  Synthetic construct
tcggtcagtcaggagtatttagc